SEQUENCE LISTING (20 sequences)

SEQ ID NO: 1 - DNA, 20 bp, Artificial sequence
  Synthetic single strand oligonucleotide
cgtggcagctcgagataacg

SEQ ID NO: 2 - DNA, 21 bp, Artificial sequence
  Synthetic single strand oligonucleotide
cggtgccatagtcaagccaaa

SEQ ID NO: 3 - DNA, 29 bp, Artificial sequence
  Synthetic single strand oligonucleotide
aggtaccgatttaatagctccatgtcaac

SEQ ID NO: 4 - DNA, 29 bp, Artificial sequence
  Synthetic single strand oligonucleotide
aggtaccgtctagaaagaaggattacctc

SEQ ID NO: 5 - protein, 7 aa, Artificial sequence
  peptide derived from chit36
FGLTMAP

SEQ ID NO: 6 - protein, 7 aa, Artificial sequence
  peptide derived from chit36
GYWENWD

SEQ ID NO: 7 - protein, 7 aa, Artificial sequence
  peptide derived from chit36
YDMQVPG

SEQ ID NO: 8 - DNA, 20 bp, Artificial sequence
  Synthetic single strand oligonucleotide
ggntaytgggaraaytggga

SEQ ID NO: 9 - DNA, 20 bp, Artificial sequence
  Synthetic single strand oligonucleotide
ccnggnacytgcatrtcrta

SEQ ID NO: 10 - protein, 20 aa, Artificial sequence
  peptide derived from chit36
VLMGYWENWDGASNGVHPGF

SEQ ID NO: 11 - protein, 15 aa, Artificial sequence
  peptide derived from chit36
IPYDMQVPGLPAQNG

SEQ ID NO: 12 - DNA, 1345 bp, Trichoderma harzianum
gttctgcttcataagtctatgatctgatctatactttctcttatttttccaatacctaatttaccatttttacttcatttcatatcttatcaggcattcagtcatgacacgccttcttgacgccagatttctgctgctgcctgccatcgcatcgacgttaattggcaccgcttctgcacaaaatgcaacatgtgcactcaagggaaaaccggccggcaaagtcctgatgggatattgggagaactgggacggagcatccaacggcgttcaccccggattcggctggacaccaatcgaaaaccctattattaaacagaatggctacaatgtgatcaacgctgctttccccgttattctgtcagatggcacagtgctgtgggaaaacgacatggctcctgatactacgattgcaacaccggccgagatgtgtgaggctaaagcagctggagcaacaattcttttgtcaatcggaggtgcttctgctggcatagacctcagctctagtacagtcgccgacaagtttgtcgcgaccattgtaccaatcttgaagcagtacaactttgacggtattgatatcgacattgagactggtttgaccggcagcggcaatatcaacacgctttccacatcccaggccaacttgattcgcatcattgatggtgttcttgctgcgatgccttcaaactttggcttgactatggcaccggagacaccatatgttacaggcggtagcgtcacgtatggctctatttggggatcatacctgccaatcattcaaaaatatatccaaaacggccgactgtggtggctcaacatgcaatactacaacgacgactactacggttgctcaggcgactcatacgcagccggcactgtcgcgggatttattgctcagactgattgcttgaatgcaggacttactgtccaaggcaccacaatcaagatcccatacgacatgcaagtacccggcctacctgcacaaaatggagctggcgggggctatatgaacccaagcttagtaggacaagcatgggaccactacaacggtgctctgaaaggcttgatgacgtggtcaatcaattgggatggagcgggtaactggacctttggcgacaacttgcttactcgcattggttagaaatagaataaaggaggaagagtttgcatttaagtcagtatatatcttgactttattccggagagataccatgtttgcacgatgtatgatacaccttttagcagtatatagttggactgactatttagtctatgtatataagtaaatagcttgatagtcttggatgaattcgagcagtttatatgaaaaaaaaaaaaaaaaaaaaaa

SEQ ID NO: 13 - protein, 344 aa, Trichoderma harzianum
MTRLLDARFLLLPAIASTLIGTASAQNATCALKGKPAGKVLMGYWENWDGASNGVHPGFGWTPIENPIIKQNGYNVINAAFPVILSDGTVLWENDMAPDTTIATPAEMCEAKAAGATILLSIGGASAGIDLSSSTVADKFVATIVPILKQYNFDGIDIDIETGLTGSGNINTLSTSQANLIRIIDGVLAAMPSNFGLTMAPETPYVTGGSVTYGSIWGSYLPIIQKYIQNGRLWWLNMQYYNDDYYGCSGDSYAAGTVAGFIAQTDCLNAGLTVQGTTIKIPYDMQVPGLPAQNGAGGGYMNPSLVGQAWDHYNGALKGLMTWSINWDGAGNWTFGDNLLTRIG

SEQ ID NO: 14 - DNA, 1939 bp, Trichoderma harzianum
atcccaggcactgggaaatggcaagggatgaaagtaaaggagtgaatgagatgttgagggaaatcttaccagggaatgaatcagtgacagaagggcaacgtgtatggcgaccacgaggttagtgcctactttaaaatgtgcgaaggcaaaatttgagagggcttcactatcgaatgagaggtttcagttttattgaagcatatccgaaaacccaattagcaacatcgtgaatgaactttgactttgacgacttaagatcctgcacttcgatatgactgtaccgagaggtgtaaaaattatgaacttgtgagaacgcttattgggctgtttaagtgtttcttaattgctgacctttgtttctccctctctgtatattgaaagggtcctcgattctgcatccctgcgcaacagccgcgtgtgtacttcacattgatcgtttgtattctttgtttcgcagtaatactgcaattgctttcttcttttgtataactagtttgaaagataaatgaataaaagatgatgaaaaggagggttgaaaagattaaaaggttttggtttccaagaaagctctgcttcgcactcaaagcgctaacgttacaacctggtccctacgctcactcggtgcttgtaccaaatgaggtactgcatataattccgtgcggttaaagtagagattaatggctaaagggaagcctaaagtgagtgtagcataccttgacgtttatccaagaaaggaaagtactgctgccgctaatcgtaacaacacaacaaaagaccgattttgcatgttgtggatcccggcttggaagcccaagacggtaggcgaaggcagagagatttctgccgttggtcagtgcacggctaccgtataccatgggacgaatttgttggtgtcaagtcgtatttatttgttcactctgtgcactcattcttttgtatatgtttttttctttcagtatgtagcatttttgattagcataatattgtcatgttctatctgggtggcaataaaggcacctgctacagtaacagcacgtgcaagacttcttgcgccaagttgcttcgcgcctgatgttacatgctctcgagctgctaaagcattgatgtaacccatgactaagtcacaagtttcaaacccatactagtgccaattattgtagcgtattactccatacttgctcttataccaagaaaagcggggtctccgccgccattgcgattacagccccctaacactgctaacttccatcttcaggcgctgaaggagcgtttattaaaccactgactgccctagattacgtactctatgttgctgcaaaatcctacgttacgcgcctacgaggggggcagcattctattccaccttcctattaatgcatgaaacttcatattagacaagataagtgattttctttcatcctttatcaggctggaaggcaccgaaaggcgctgaatatgagacacatgcgccaagctttcgattccttgtactggagggagaagaaaaaaaaaaagtgtggccctatgtatcgcagcaacttcagcgctcttctttgcattgggcagagtcgcggtggaagaggaatgaacttgtagagcagatgagcaactcacaaatcttcatttatccaacgacataatgttaggaatgtagggttatcatttgacaaaatgggtgatccatagaatggaatttagctcaccagactaagtcatgatactaccttcttagaataggcaaggaaggagaaagagatatatgaagccaagaaatcaaaggactatgctctataaaagcgctctatatgccaaccaattcgtatgagttctcatcgtttgtcaacaaacactttgacctgatctattcctcctcttgacttttgagtatataattttgactattattaccttattttatatcttataaggcatcctaatc

SEQ ID NO: 15 - DNA, 5 bp, Artificial sequence
  Putative tataa-box derived from the upstream sequence of the chit 36 gene
tataa

SEQ ID NO: 16 - DNA, 5 bp, Artificial sequence
  Putative ccaat-box derived from the upstream sequence of the chit 36 gene
ccaat

SEQ ID NO: 17 - DNA, 6 bp, Artificial sequence
  Consensus motif for the CreA/Cre1 carbon catabolic repressor
syggrg

SEQ ID NO: 18 - DNA, 6 bp, Artificial sequence
  Consensus motif for binding of A. nidulans global nitrogen regulator
hgatar

SEQ ID NO: 19 - DNA, 6 bp, Artificial sequence
  Consensus motif for binding of A. nidulans AbaA developmental regulator
cattcy

SEQ ID NO: 20 - DNA, 3 bp, Artificial sequence
  motif found in the URSs of both ech42 and prb1 genes
myc

(19) United States
(12) Patent Application Publication
(10) Pub. No.: US 2004/0121442 A1
(43) Pub. Date: Jun. 24, 2004

(54) Fungal chitinase, polynucleotide sequences encoding same, promoters of same, and uses thereof

(76) Inventors: Ilan Chet, Nes Ziona (IL); Ada Viterbo, Rehovot (IL)

Correspondence Address: Anthony Castorina, G E Ehrlich, Suite 207, 2001 Jefferson Davis Jefferson, Arlington, VA, 22202, US

(21) Appl. No.: 10/475,853

(22) PCT Filed: May 5, 2002
(86) PCT No.: PCT/IL02/00351

(51) Int. Cl. C12N 9/24; C07H 21/04

(52) U.S. Cl. 435/200; 435/69.1; 435/320.1; 435/325; 536/23.2

(57) ABSTRACT

A method of preventing or treating a disease or a condition associated with a chitin-containing organism in an individual, the method comprising administering to the individual a therapeutically effective amount of a pharmaceutical composition including as an active ingredient a polypeptide displaying an endochitinase activity.

### FIELD AND BACKGROUND OF THE INVENTION

The present invention is of recombinant fungal chitinases, polynucleotides encoding such chitinases, and uses thereof in treatment of human diseases caused by chitin-containing organisms, such as the fungus Candida albicans, and treatment of plant diseases caused by or associated with chitin-containing pathogens, such as fungal pathogens. The present invention is further of polynucleotides encoding regulatory sequences of genes encoding fungal chitinases and uses thereof in reducing susceptibility of plants to damage from stress conditions.

Chitin-containing organisms, such as fungi, helminths, arthropods, and protozoans include pathogens responsible for causing a very broad range of diseases in humans, including many widespread, highly debilitating and/or lethal diseases of major clinical impact. Such organisms also include numerous pathogenic and pest species of plants, livestock and animals responsible for routinely causing enormous damage to agricultural productivity.

Hence, novel and improved agents capable of controlling such harmful organisms are urgently required.

A variety of infectious diseases, including widespread and/or life-threatening diseases of major clinical impact in man are caused by chitin-containing organisms, such as fungi, protozoans, helminths, etc. Fungal infections, or mycoses, despite the existence of anti-fungal therapeutics, are increasingly responsible for life-threatening disorders (Georgopapadakou et al., 1995. Trends Microbiol. 3:98).

Types of fungi that can cause severe infectious diseases in man include fungi of the genera Aspergillus, Cryptococcus, Coccidioides, Paracoccidioides, Blastomyces, Sporothrix, and Histoplasma.

The most frequent fungal infection in humans is caused by Candida albicans. This organism is a common commensal organism of the oral and vaginal mucosae but can become a pathogen on damaged skin in severely ill patients, and in patients receiving broad-spectrum antibiotics when the local microbial ecology is disturbed. Extreme consequences of Candida infection include pneumonia, endocarditis, septicaemia and death.

Fungal pathogens, are particularly dangerous in immunocompromised individuals, such as patients with AIDS, patients undergoing chemotherapy, and immunosuppressed organ transplant patients where opportunistic infections such as Pneumocystis carinii are responsible for significant morbidity and mortality. For example, it has been estimated that 58-81% of all AIDS patients contract a fungal infection at some time during the prodromal stage or after developing AIDS, and that 10-20% have died as a direct consequence of fungal infections. Major mycoses related to AIDS include candidiasis, the yeast infection cryptococcosis, histoplasmosis, and coccidioidomycosis (dimorphic fungi). Severe mycoses related to AIDS include penicilliosis, blastomycosis, paracoccidioidomycosis, sporotrichosis, aspergillosis, mucormycosis, and nocardiosis. Cutaneous fungal infections related to AIDS include seborrheic dermatitis, dermatophytosis, trichosporonosis, and alteraariosis.

In addition to directly causing diseases, fungal antigens function as immunosuppressants thereby creating conditions suitable for opportunistic infections or aggravation of pre-existing infections, particularly in immunocompromised individuals. For example, fungal circulating antigens, such as mannan in candidiasis, and glucuronoxylomannan in cryptococcosis may be important cofactors in AIDS.

Aspergillosis is a common fungal infection in immunodepressed patients, occurring at a rate as high as 70% in patients with leukemia after 30 days of neutropenia.

Histoplasmosis, an infection of macrophages, is a disease similar in severity to tuberculosis. In normal hosts acute pulmonary infection is often accompanied by cough and chest pain, myalgia and weight loss, and in immunocompromised hosts disseminated histoplasmosis may develop, accompanied by fever, hepatosplenomegaly, anaemia, leucopenia, thrombocytopenia and pneumonia.

Other severe human diseases caused by non-fungal chitin-containing microorganisms include toxoplasmosis, malaria, Chagas' disease, and sleeping sickness. Severe human diseases caused by helminths include schistosomiasis, trichinosis, filariasis and ochocerciasis.

Thus, diseases caused by chitin-containing organisms are a major clinical problem.

There exists a wide variety of chitin-containing plant pathogens, such as fungi, insects and nematodes.

Fungal phytopathogens include species from genera such as Fusarium, Pythium, Phytophthora, Verticillium, Rhizoctonia, Macrophomina, Thielaviopsis, Sclerotinia and numerous others. Such pathogens cause plant diseases such as pre- and post-emergence seedling damping-off, hypocotyl rots, root rots, crown rots, vascular wilts and a variety of other diseases.

Nematode phytopathogens also include species from numerous generas, including, for example, Meloidogyne, Heterodera, Ditylenchus and Pratylenchus, and cause diseases including root galls, root rot, lesions, “stubby” root, stunting, and various other rots and wilts associated with increased infection by pathogenic fungi. Some nematodes (e.g., Trichodorus, Lonaidorus, Xiphenema) furthermore are vectors of viral diseases in a number of commercially important plants including prune, grape, tobacco and tomato.

Chitin, the second most abundant organic substance after cellulose is an important component of the cell wall of fungi, the exoskeleton of arthropods and the outer cell wall of numerous parasites, such as protozoans and helminths, where it serves to provide protection from mechanical and chemical stress from the environment and to provide structural and morphological support. In most structures, chitin is associated with other substances.

Chitin is an unbranched polysaccharide polymer consisting of N-acetyl-D-glucosamine (GluNAc) units joined by β-1,4 glycosidic linkages whose chain length of N-acetylglucosamine polymers ranges from about 100 to 8,000 units. Most fungi contain considerable amounts of chitin and, in fungal cell walls, this polymer is often associated with β-1,3/β-1,6 glucan, polymers of glucose with β-1,3 and β-1,6 linkages. Such polymers assemble laterally to form microfibrils which are stabilized by strong hydrogen bonds between the amine group of sugar in one chain and the carbonyl group of sugar in a neighbouring chain. Three crystallographic forms of chitin have been identified. In α-chitin, the most abundant form in fungi and arthropods, adjacent chains are oriented antiparallel to each other; in β-chitin the chains are oriented parallel to each other, whilst in γ-chitin one pair of chains are parallel to each other and the third chain is anti- parallel to the pair. The microfibrils in fungi and crustaceans usually show a diameter of 20-25 nm.

Chitin is insoluble in water, dilute acids and bases but can be broken down enzymatically by chitinases, yielding degradation products such as soluble monomers or multimers of GluNAc.

Chitinases are a class of hydrolytic enzymes which degrade chitin by endolytic or exolytic mechanisms. Chitinases include exochitinases (β-1,4-N-acetylglucosoaminidases) catalyzing cleavage of diacetylchitobiose units from the non-reducing end of the polysaccharide; and specific endochitinases which cleave glycosidic linkages randomly along the chitin chain, leading mainly to production of diacetylchitobiose and to smaller quantities of triacetylchitotriose. Many of the chitinases found in plants cleave the internal β-1,4 glycosidic linkages in the chitin molecules to liberate oligomers of at least 3 GluNAc units.

The need for organisms to protect themselves from chitin-containing organisms, such as fungi, is demonstrated in the fact that many species of bacteria, fungi, plants, fish, mammals, etc., produce chitinases.

In humans, studies suggest chitinases may play essential roles in immunity against chitin-containing pathogens, such as fungi, helminths, protozoans, etc. For example, chitinase activity has been demonstrated in human leukocytes (Escott et al., 1995. Infect. Immun. 63:4770), a chitinase (4-methylumbelliferyl-tetra-N-acetylchitotetraoside hydrolase) has been isolated from human serum and rat liver (Overdijk et al., 1994. Glycobiology 4:797), a human chitotriosidase has been isolated from human spleen (Renkema et al., 1995. J. Biol. Chem. 270:2198), and human macrophage cDNA encoding a chitinase has been cloned (Boot et al., 1995. J. Biol. Chem. 270:26252).

Since chitin is absent in plant and vertebrate cells, but is present in the very large variety of chitin-containing organisms that are pathogenic in plants and vertebrates, such as humans, it serves as an attractive target for selectively attacking these type of pathogens. Indeed, disruption of chitin-containing fungal cell wall and parasite membrane has been a useful therapeutic strategy against fungi and parasites. For example, in mammals, the anti-fungal agents amphotericin B and fluconazole exert their anti-fungal activity by affecting membrane steroids.

Thus, chitinases, enzymes specialized in degrading chitin, represent a potentially powerful tool for controlling chitin-containing pathogens, such as fungi. Certain species of fungi constitute an attractive source from which to derive recombinant chitinases for use against fungal phytopathogens. For example, Trichoderma harzianum, a filamentous soil fungus known as an effective biocontrol agent of several plant pathogenic fungi, has been shown to exert anti-fungal activity via chitinases. The chitinolytic system in T. harzianum consists of at least six distinct enzymes, two N-acetylglucosaminidases and four endochitinases (Haran S., et al., 1995. Mycol. Res. 99:441). A number of chitinolytic enzymes from T. harzianum have been purified (De La Cruz J. et al., 1992. Eur. J. Biochem. 206:859; Harman G E. et al., 1993. Phytopathology 83:313; Lorito M. et al., 1993. Phytopathology 83:302; Garcia I. et al., 1994. Curr. Genet. 27:83; Draborg H. et al., 1995. Biochem, and Molec. Biol. Int. 36:781; Peterbauer C K. et al., 1996. Curr. Genet. 30:325) and their antifungal activity has been demonstrated against a variety of plant pathogens (Carsolio et al., 1999, Kulling et al., 2000, Dana et al., 2001). To date, the chitinase genes which have been isolated from Trichoderma strains are the ones coding for CHIT102 and CHIT73 (Draborg H. et al., 1995. Biochem, and Molec. Biol. Intern. 36:781), CHIT42 (Garcia I. et al., 1994. Curr. Genet. 27:83) and CHIT33 (Limon M C. et al., 1995. Curr. Genet. 28:478).

Several prior art approaches have been employed in order to attempt to use recombinant fungal chitinases as anti-fungal agents.

One approach has employed transgenic expression of a recombinant T. harzianum endochitinase in E. coli and used such transformed bacteria to protect plants from Sclerotium rolfsii infection (Chet I. et al., 1993. Int Sym Chitin Enzymol, Senigallia (Italy)).

Another approach has used transgenic expression of recombinant T. harzianum chet42 endochitinase in tobacco and potato plants to protect such transgenic tobacco plants from Alternaria alternata or Botrytis cinerea infection, or to protect such transgenic potato plants from Rhizoctonia solani or A. alternata infection (Lorito M. et al., 1998. Proc Natl Acad Sci USA. 95:7860). In this approach, however, high levels of transgene expression in tobacco were required to yield significant protection from pathogens and only 5-10% of transgenic plants displayed high levels of disease resistance.

Yet another approach has utilized transgenic expression of recombinant T. harzianum chet42 endochitinase in apple plants in an attempt to protect such transgenic plants from Venturia inaequalis infection (Bolar J P. et al., 2000. Phytopathology 90:72). In this approach, however, expression of even moderate levels of the enzyme caused significant reductions in plant vigor.

Still another approach has employed transgenic expression of a Trichoderma atroviride (formerly T. harzianum P1) exochitinase in apple plants in an attempt to protect such transgenic plants from V. inaequalis infection (Prakash J P. et al., 2001. Transgenic Research 10:533). In this approach however, only slight protection was afforded by the exochitinase.

An additional approach has used transgenic combined expression of both an endochitinase and an exochitinase from T. atroviride in apple plants in an attempt to protect such transgenic plants from V. inaequalis infection (Prakash J P. et al., 2001. Transgenic Research 10:533). In this approach however, only incomplete and/or inconsistent protection of transgenic plants from infection by the pathogen was afforded by the transgenes.

Importantly, none of the aforementioned prior art approaches was shown to be effective against the fungal pathogen Fusarium.

Thus, all prior art approaches have failed to provide an adequate solution for providing recombinant fungal chitinases capable of effectively and consistently functioning as effective agents against chitin-containing organisms such as pathogenic fungi.

There is thus a widely recognized need for, and it would be highly advantageous to have, a fungal chitinase devoid of the above limitation.

### SUMMARY OF THE INVENTION

According to the present invention there is provided an isolated polypeptide displaying an endochitinase activity and comprising an amino acid sequence being at least 84% similar to SEQ ID NO: 13 or a portion thereof, as determined using the Standard protein-protein BLAST [blastp] software of the NCBI.

According to another aspect of the present invention there is provided an enzymatic composition comprising a polypeptide displaying an endochitinase activity, the polypeptide comprising an amino acid sequence being at least 84% similar to SEQ ID NO: 13 or a portion thereof, as determined using the Standard protein-protein BLAST [blastp] software of the NCBI.

According to still another aspect of the present invention there is provided an isolated polynucleotide comprising a nucleic acid sequence being at least 84% identical to SEQ ID NO: 12 or a portion of SEQ ID NO: 12, as determined using the Standard nucleotide-nucleotide BLAST [blastn] software of the NCBI, the nucleic acid sequence encoding a polypeptide displaying an endochitinase activity.

According to yet another aspect of the present invention there is provided an isolated polynucleotide comprising a nucleic acid sequence being at least 30% identical to SEQ ID NO: 14 or a portion thereof, as determined using the Standard nucleotide-nucleotide BLAST [blastn] software of the NCBI, the nucleic acid sequence being capable of inducing a specific change in a level of expression of a reporter gene under the regulatory control of the nucleic acid sequence in a cell in response to exposure of the cell to a specific environmental condition.

According to an additional aspect of the present invention there is provided a nucleic acid construct comprising a nucleic acid sequence being at least 84% identical to SEQ ID NO: 12, or a portion of SEQ ID NO: 12, as determined using the Standard nucleotide-nucleotide BLAST [blastn] software of the NCBI, the nucleic acid sequence encoding a polypeptide displaying an endochitinase activity.

According to still an additional aspect of the present invention there is provided a nucleic acid construct comprising a nucleic acid sequence being at least 30% identical to SEQ ID NO: 14, or a portion thereof, as determined using the Standard nucleotide-nucleotide BLAST [blastn] software of the NCBI, the nucleic acid sequence being capable of inducing a specific change in a level of expression of a reporter gene under the regulatory control of the nucleic acid sequence in a cell in response to exposure of the cell to a specific environmental condition.

According to yet an additional aspect of the present invention there is provided a host cell comprising a nucleic acid construct, the nucleic acid construct comprising a nucleic acid sequence being at least 84% identical to SEQ ID NO: 12, or a portion of SEQ ID NO: 12, as determined using the Standard nucleotide-nucleotide BLAST [blastn] software of the NCBI, the nucleic acid sequence encoding a polypeptide displaying an endochitinase activity.

According to a further aspect of the present invention there is provided a host cell comprising a nucleic acid construct, the nucleic acid construct comprising a nucleic acid sequence being at least 30% identical to SEQ ID NO: 14, or a portion thereof, as determined using the Standard nucleotide-nucleotide BLAST [blastn] software of the NCBI, the nucleic acid sequence being capable of inducing a specific change in a level of expression of a reporter gene under the regulatory control of the nucleic acid sequence in a cell in response to exposure of the cell to a specific environmental condition.

According to still a further aspect of the present invention there is provided a method of preventing or treating a disease or a condition associated with a chitin-containing organism in an individual, the method comprising administering to the individual a therapeutically effective amount of a pharmaceutical composition including as an active ingredient a polypeptide displaying an endochitinase activity and being at least 84% similar to SEQ ID NO: 13 or a portion thereof, as determined using the Standard protein-protein BLAST [blastp] software of the NCBI, the pharmaceutical composition further including a pharmaceutically acceptable carrier or diluent.

According to yet a further aspect of the present invention there is provided a pharmaceutical composition comprising as an active ingredient a polypeptide comprising an amino acid sequence being at least 84% similar to SEQ ID NO: 13 or a portion thereof, as determined using the Standard protein-protein BLAST [blastp] software of the NCBI, and a pharmaceutically acceptable carrier or diluent, the polypeptide displaying an endochitinase activity.

According to another aspect of the present invention there is provided a method of preventing or treating a disease or condition associated with a chitin-containing organism in a plant, the method comprising contacting the plant with a composition including as an active ingredient a polypeptide displaying an endochitinase activity and being at least 84% similar to SEQ ID NO: 13 or a portion thereof, as determined using the Standard protein-protein BLAST [blastp] software of the NCBI.

According to still another aspect of the present invention there is provided an agronomic composition comprising as an active ingredient a polypeptide displaying an endochitinase activity, the polypeptide comprising an amino acid sequence being at least 84% similar to SEQ ID NO: 13 or a portion thereof, as determined using the Standard protein-protein BLAST [blastp] software of the NCBI.

According to yet another aspect of the present invention there is provided a method of preventing or treating a disease or condition associated with a chitin-containing organism in a plant, the method comprising expressing within the plant an exogenous polypeptide displaying an endochitinase activity and being at least 84% similar to SEQ ID NO: 13 or a portion thereof, as determined using the Standard protein-protein BLAST [blastp] software of the NCBI.

According to an additional aspect of the present invention there is provided a method of preventing or reducing susceptibility of a plant to cold damage, the method comprising expressing within the plant an exogenous polypeptide displaying an endochitinase activity and being at least 84% similar to SEQ ID NO: 13 or a portion thereof, as determined using the Standard protein-protein BLAST [blastp] software of the NCBI.

According to still an additional aspect of the present invention there is provided a plant, a plant tissue or a plant seed comprising an exogenous polynucleotide, the exogenous polynucleotide comprising a nucleic acid sequence being at least 84% identical to SEQ ID NO: 12 or a portion of SEQ ID NO: 12, as determined using the Standard nucleotide-nucleotide BLAST [blastn] software of the NCBI, the nucleic acid sequence encoding a polypeptide displaying an endochitinase activity.

According to yet an additional aspect of the present invention there is provided a composition for disinfesting chitin-containing organisms, the composition comprising as an active ingredient a polypeptide displaying an endochitinase activity, the polypeptide comprising an amino acid sequence being at least 84% similar to SEQ ID NO: 13 or a portion thereof as determined using the Standard protein-protein BLAST [blastp] software of the NCBI.

According to a further aspect of the present invention there is provided A method of inducing a specific change in a level of expression of a gene product in a cell in response to an exposure of the cell to a specific environmental condition, the method comprising expressing the gene product in the cell under the regulatory control of an exogenous polynucleotide comprising a nucleic acid sequence being at least 30% identical to SEQ ID NO: 14 or a portion thereof, as determined using the Standard nucleotide-nucleotide BLAST [blastn] software of the NCBI.

According to still a further aspect of the present invention there is provided a method of reducing the susceptibility of a plant to a damage resulting from an exposure to a specific environmental condition, the method comprising expressing a gene product in the plant under the regulatory control of an exogenous polynucleotide comprising a nucleic acid sequence being at least 30% identical to SEQ ID NO: 14 or a portion thereof, as determined using the Standard nucleotide-nucleotide BLAST [blastn] software of the NCBI, the gene product being capable of reducing the susceptibility of the plant to the damage resulting from the exposure to the specific environmental condition.

According to yet a further aspect of the present invention there is provided a plant, a plant tissue or a plant seed comprising an exogenous polynucleotide, the exogenous polynucleotide comprising a nucleic acid sequence being at least 30% identical to SEQ ID NO: 14 or a portion thereof, as determined using the Standard nucleotide-nucleotide BLAST [blastn] software of the NCBI, the nucleic acid sequence being capable of inducing a specific change in a level of expression of a reporter gene under the regulatory control of the nucleic acid sequence in a cell in response to exposure of the cell to a specific environmental condition.

According to further features in preferred embodiments of the invention described below, the fungus is selected from the group consisting of Botrytis cinerea, Fusarium oxysporum, Sclerotium rolfsii and Candida albicans.

According to still further features in the described preferred embodiments, the disease or condition is dermal or mucosal and further wherein the carrier or diluent is formulated for topical administration.

According to still further features in the described preferred embodiments, the chitin-containing organism is Candida albicans.

According to still further features in the described preferred embodiments, the carrier or diluent is formulated for topical administration.

According to still further features in the described preferred embodiments, the method of preventing or treating a disease or condition associated with a chitin-containing organism in a plant, wherein the composition further includes a diluent.

According to still further features in the described preferred embodiments, the enzymatic composition, further comprising a polypeptide displaying an exochitinase activity.

According to still further features in the described preferred embodiments, the pharmaceutical composition further includes as an active ingredient a polypeptide displaying an exochitinase activity.

According to still further features in the described preferred embodiments, the pharmaceutical composition, further comprising as an active ingredient a polypeptide displaying an exochitinase activity.

According to still further features in the described preferred embodiments, the agronomic composition, further comprising as an active ingredient a polypeptide displaying an exochitinase activity.

According to still further features in the described preferred embodiments, the agronomic composition further comprises an agronomically acceptable carrier or diluent.

According to still further features in the described preferred embodiments, the isolated polypeptide is characterized by an apparent molecular weight of about 33 kDa, as determined via 12% SDS-PAGE following deglycosylation.

According to still further features in the described preferred embodiments, the polypeptide displaying the endochitinase activity is characterized by an apparent molecular weight of about 33 kDa, as determined via 12% SDS-PAGE following deglycosylation.

According to still further features in the described preferred embodiments, the exogenous polypeptide is characterized by an apparent molecular weight of about 33 kDa, as determined via 12% SDS-PAGE following deglycosylation.

According to still further features in the described preferred embodiments, the isolated polypeptide is characterized by a pI selected from a range of about 4.5 to about 4.9.

According to still further features in the described preferred embodiments, the polypeptide displaying the endochitinase activity is characterized by a pI selected from a range of about 4.5 to about 4.9.

According to still further features in the described preferred embodiments, the exogenous polypeptide is characterized by a pI selected from a range of about 4.5 to about 4.9.

According to still further features in the described preferred embodiments, the endochitinase activity is optimal at a pH of about 4.5.

According to still further features in the described preferred embodiments, the endochitinase activity is optimal at a temperature selected from a range of about 40° C. to about 53° C.

According to still further features in the described preferred embodiments, the isolated polypeptide comprises a signal peptide.

According to still further features in the described preferred embodiments, the polypeptide displaying the endochitinase activity comprises a signal peptide.

According to still further features in the described preferred embodiments, the signal peptide is for extracellular secretion of the isolated polypeptide.

According to still further features in the described preferred embodiments, the exogenous polypeptide comprises a signal peptide.

According to still further features in the described preferred embodiments, the signal peptide is for extracellular secretion of the polypeptide displaying the endochitinase activity.

According to still further features in the described preferred embodiments, the signal peptide is for extracellular secretion of the exogenous polypeptide.

According to still further features in the described preferred embodiments, the signal peptide comprises amino acid residues 1-16 of SEQ ID NO: 13.

According to still further features in the described preferred embodiments, the isolated polypeptide exhibits an activity against a chitin-containing organism.

According to still further features in the described preferred embodiments, the polypeptide displaying the endochitinase activity exhibits an activity against the chitin-containing organism.

According to still further features in the described preferred embodiments, the exogenous polypeptide exhibits an activity against the chitin-containing organism.

According to still further features in the described preferred embodiments, the activity against the chitin-containing organism is selected from the group consisting of inhibition of growth of the chitin-containing organism, killing of the chitin-containing organism and inhibition of reproduction of the chitin-containing organism.

According to still further features in the described preferred embodiments, the activity against the chitin-containing organism is selected from the group consisting of inhibition of growth of the chitin-containing organism, killing of the chitin-containing organism and inhibition of reproduction of the chitin-containing organism.

According to still further features in the described preferred embodiments, the chitin-containing organism is a fungus.

According to still further features in the described preferred embodiments, the chitin-containing organism is associated with pathogenesis of the disease or condition.

According to still further features in the described preferred embodiments, the fungus is selected from the group consisting of Botrytis cinerea, Fusarium oxysporum and Sclerotium rolfsii.

According to still further features in the described preferred embodiments, the portion of SEQ ID NO: 12 is selected from the group consisting of nucleotides 1-1138 of SEQ ID NO: 12, nucleotides 104-1345 of SEQ ID NO: 12, nucleotides 104-1138 of SEQ ID NO: 12, nucleotides 152-1345 of SEQ ID NO: 12, and nucleotides 152-1138 of SEQ ID NO: 12.

According to still further features in the described preferred embodiments, the polypeptide is at least 84% similar to SEQ ID NO: 13, or a portion of SEQ ID NO: 13.

According to still further features in the described preferred embodiments, the isolated polynucleotide is selected from the group consisting of a genomic polynucleotide, a complementary polynucleotide and a composite polynucleotide.

According to still further features in the described preferred embodiments, the exogenous polynucleotide is selected from the group consisting of a genomic polynucleotide, a complementary polynucleotide and a composite polynucleotide.

According to still further features in the described preferred embodiments, the composition for disinfesting chitin-containing organisms further comprises a carrier or diluent.

According to still further features in the described preferred embodiments, the composition further includes as an active ingredient a polypeptide displaying an exochitinase activity.

According to still further features in the described preferred embodiments, the composition for disinfesting chitin-containing organisms, further comprising as an active ingredient a polypeptide displaying an exochitinase activity.

According to still further features in the described preferred embodiments, the polypeptide displaying the exochitinase activity is EXC-1.

According to still further features in the described preferred embodiments, the specific environmental condition is a stress condition and whereas the specific change is an increase in the level of expression of the reporter gene.

According to still further features in the described preferred embodiments, the specific environmental condition is an elevated glucose concentration and whereas the specific change is a decrease in the level of expression of the reporter gene.

According to still further features in the described preferred embodiments, the specific environmental condition is an elevated glucose concentration and whereas the specific change is a decrease in the level of expression of the gene product.

According to still further features in the described preferred embodiments, the specific environmental condition is a stress condition and whereas the specific change is an increase in the level of expression of the gene product.

According to still further features in the described preferred embodiments, the cell is a T. harzianum cell or a P. pastoris cell.

According to still further features in the described preferred embodiments, the host cell is a Trichoderma harzianum cell or a Pichia pastoris cell.

According to still further features in the described preferred embodiments, the cell is a plant cell.

According to still further features in the described preferred embodiments, the host cell is a plant cell.

According to still further features in the described preferred embodiments, the portion is amino acid residues 17 to 344 of SEQ ID NO: 13.

According to still further features in the described preferred embodiments, the stress condition is selected from the group consisting of a temperature extreme, an elevated chitin concentration, a chitin-containing organism, osmotic stress and nitrogen starvation.

According to still further features in the described preferred embodiments, the temperature extreme is a temperature no greater than about 4° C. or a temperature no lower than about 40° C.

According to still further features in the described preferred embodiments, the elevated glucose concentration is no lower than about 50 g/L.

According to still further features in the described preferred embodiments, the elevated chitin concentration is no lower than about 2 g/L.

According to still further features in the described preferred embodiments, the reporter gene encodes a structural sequence of chit36 or gfp.

According to still further features in the described preferred embodiments, the gene product is a chit36 gene product or a gfp gene product.

According to still further features in the described preferred embodiments, the polypeptide displays an endochitinase activity and comprises an amino acid sequence being at least 84% similar to SEQ ID NO: 13 or a portion thereof, as determined using the Standard protein-protein BLAST [blastp] software of the NCBI.

According to still further features in the described preferred embodiments, the gene product is endogenous or exogenous to the cell.

According to still further features in the described preferred embodiments, the gene product is endogenous or exogenous to the plant.

According to still further features in the described preferred embodiments, the gene product is a messenger RNA or a polypeptide.

According to still further features in the described preferred embodiments, the specific environmental condition is a stress condition or an elevated glucose concentration.

According to still further features in the described preferred embodiments, the portion is nucleotides 1110-1139 of SEQ ID NO: 14.

According to still further features in the described preferred embodiments, the nucleic acid sequence is a promoter, an enhancer or a suppressor.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are schematic diagrams depicting PCR amplification of pRL-36 (FIG. 1a) and pAN7 (FIG. 1b) specific sequences from genomic DNA of T. harzianum transformants. Primers used for PCR amplification are indicated by numbered arrows; primer 1, (SEQ ID NO: 1), primer 2, (SEQ ID NO: 2), primer 3, (SEQ ID NO: 3), primer 4, (SEQ ID NO: 4). ppki1: pki1 promoter, pGPD: GPD promoter, hgh: hygromycin B phosphotransferase gene.

FIG. 2 is a Blast sequence homology search diagram depicting comparison of the amino acid sequences of CHIT36 from T. harzianum Rifai TM with that of CHIT33 from T. harzianum Rifai CECT2413 and that of the putative chitinase CAB69724 from S. coecicolor.

FIGS. 3a-b are fluorescence photographs depicting PCR amplification of pAN7 specific (FIG. 2a) and pRL36 specific (FIG. 2b) sequences from genomic DNA of T. harzianum transformants. (+): positive control.

FIG. 4 is a photograph depicting an endochitinase activity assay of T. harzianum transformants. A12: wild-type T. harzianum grown in chitin supplemented medium, B12: wild-type T. harzianum grown in glucose rich medium, [rows A, B, C, D]×[columns 1-6]: tested transformants.

FIG. 5 is a photograph of a Southern Blot analysis depicting integration of ppki1-chit36 transgene into the genome of T. harzianum transformants. Ten micrograms of DNA were digested with EcoRI which cleaves the pRL36 plasmid once, and probed with DNA the chit36 ORF.

FIGS. 6a-b are photographs depicting Northern blot analysis of chit36 RNA expression in mycelia of T. harzianum chit36 transformants. FIG. 6a depicts hybridization of total RNA (15 μg) using the full-length ORF sequence of chit36 as a probe. FIG. 6b depicts methylene blue staining of the filter as loading control. WT-G and WT-C: wild-type T. harzianum grown in glucose rich or chitin supplemented medium, respectively.

FIGS. 7a-b are photographs depicting a Western immunoblotting assay (FIG. 7a) and an in situ gel chitinase activity assay (FIG. 7b) of culture filtrates of T. harzianum chit36 transformants. Protein from 40 μl of 40-fold concentrated culture filtrate per lane were separated via SDS-PAGE in a 12% polyacrylamide gel. WT-C and WT-G: wild-type T. harzianum grown in chitin-supplemented or glucose rich medium, respectively.

FIGS. 8a-d are microphotographs depicting almost complete inhibition of B. cinerea spore germination by treatment with culture filtrate of T. harzianum chit36 transformants. FIG. 8a-negative control treatment with culture filtrate of wild-type T. harzianum grown in glucose rich medium (0.003 O.D. units·ml⁻¹·h⁻¹), FIG. 8b—positive control treatment with culture filtrate of wild-type T. harzianum grown in chitinase supplemented medium (0.8 O.D. units·ml⁻¹·h⁻¹), FIG. 8c-treatment with culture filtrate of transformant C (0.34 O.D. units·ml⁻¹·h⁻¹). FIG. 8d-treatment with PBS only. Microphotographs were taken at ×150 magnification.

FIG. 8e is a histogram depicting almost complete inhibition of B. cinerea spore germination by treatment with culture filtrate of T. harzianum chit36 transformants. “WT-G”: negative control treatment with culture filtrate of wild-type T. harzianum grown in glucose rich medium (0.003 O.D. units·m⁻¹·h⁻¹), “WT-C”: positive control treatment with culture filtrate of wild-type T. harzianum grown in chitinase supplemented medium (0.8 O.D. units·m⁻¹·h⁻¹), “C”: treatment with culture filtrate of transformant C, “PBS”: treatment with PBS only.

FIGS. 9a-c are fluorescent photographs depicting growth of His⁺ P. pastoris transformants. FIGS. 9a-b—P. pastoris transformed with pPIC9K-36, FIG. 9c—P. pastoris transformed with empty pPIC9K vector.

FIGS. 9d-e are photographs depicting chitinase activity and specific expression of CHIT36 in culture filtrates of P. pastoris chit36 transformants separated via native PAGE. FIG. 9d is a fluorescence photograph of an in situ gel chitinase activity assay depicting chitinase activity in culture filtrates of transformants using staining with 4-MU-[GlcNAc]₂. FIG. 9e depicts a Western immunoblotting assay demonstrating the presence of secreted CHIT36 in culture filtrates of transformants. Immunoblotting was performed using anti-CHIT36 antibody diluted 1:1000. V: culture filtrate of P. pastoris transformed with empty pPIC9K vector, Ti: culture filtrate of T. harzianum grown in medium supplemented with 0.2% chitin (EXC-1 is a T. harzianum exochitinase). Lanes identified by numbers represent P. pastoris chit36 transformants.

FIGS. 10a-b are photographs of SDS-PAGE analyses depicting purification and deglycosylation of recombinant CHIT36 (rCIT36) from culture filtrate of P. pastoris chit36 transformants. FIG. 10a depicts Coomassie blue staining of culture filtrate proteins. Lane 1: culture filtrate of P. pastoris transformed with empty pPIC9K vector (50 μl supernatant of 200 ml culture), Lane 2: culture filtrate of transformant 32.1 (pPIC9K-36; 50 μl supernatant of 200 ml culture), Lane 3: low molecular weight markers, Lane 4: 5 μg of Q-Sepharose purified CHIT36. FIG. 10b depicts a Western immunoblotting assay of CHIT36 in protein in culture filtrates of P. pastoris chit36 transformants using anti-CHIT36 antibody. Lane 1: 5 μg of Q-Sepharose purified rCHIT36+500 units peptide-N-glycosidase F (PNGase F), Lane 2: culture filtrate of wild-type T. harzianum TM grown in medium supplemented with 0.2% colloidal chitin. Culture filtrate proteins were separated via 12% SDS-PAGE.

FIG. 11 is a schematic diagram depicting identification of consensus sequences within the 830 bp upstream of chit36 coding sequences comprising the chit36 promoter. Polypeptides/polypeptide sequences bound by the nucleic acid consensus sequences are indicated in parentheses.

FIG. 12 is an autoradiograph of a Northern blot depicting stress-inducible expression of chit36 mRNA in T. harzianum chit36 transformants. Lane 1: growth in glucose rich medium, Lane 2: growth in chitin supplemented medium, Lane 3: 4° C. treatment, Lane 4: 40° C. treatment, Lane 5: 2% EtOH treatment, Lane 6: 1% glucosamine treatment, Lane 7: 1% N-acetylglucosamine treatment, Lane 8: nitrogen starvation treatment.

FIGS. 13a-e are fluorescence (FIGS. 13a, 13c and 13e) and phase-contrast (FIGS. 13b, 13d and 13f) photomicrographs depicting T. harzianum transformants expressing GFP under the regulatory control of the chit36 promoter. The T. harzianum transformants were grown for 24 h in the presence of R. solani secretions from a 2-day R. solani culture (FIGS. 13a-b), following transfer to co-culture with a 2-day R. solani culture (FIGS. 13c-d) or in virgin agar (FIGS. 13e-f)

### DESCRIPTION OF THE PREFERRED EMBODIMENTS

The present invention is of recombinant fungal chitinases, compositions containing such chitinases, polynucleotide sequences encoding such chitinases and methods of using such chitinases, compositions and polynucleotides to prevent or treat diseases or conditions caused by chitin-containing organisms such as the fungus Candida albicans, in humans, and fungal phytopathogens in plants, and to disinfest chitin-containing organisms. The present invention is further of stress inducible chitinase gene promoters and methods of using such promoters to protect cells from stress damage.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings described in the Examples section hereinbelow. The invention is capable of other embodiments or of being practiced or carried out in vacuous ways. Also, it is to be understood that the phraseology and terminology employed herein is for the purpose of description and should not be regarded as limiting.

Humans, plants, such as crop plants, and animals, such as livestock, are susceptible to diseases and damage caused by a wide variety of chitin-containing pathogens, such as fungi. In humans, severe and widespread diseases, such as Candida albicans infection, are caused by fungi and are of major clinical impact. In plants, fungal diseases may cause multiple growth defects including pre- and post-emergence seedling damping-off, root-rots, crown-rots, lesions, vascular wilts and a variety of other forms of symptoms, which often result in the destruction of entire crops. Thus, agents having effective activity against chitin-containing organisms, such as pathogenic fungi, are urgently required.

Several prior art approaches have been employed in order to attempt to use recombinant fungal chitinases as anti-fungal agents.

One approach has employed transgenic expression of a recombinant T. harzianum chitinase in E. coli and used such transformed bacteria to attempt to protect plants from Scierotium rolfsii infection (Chet I. et al., 1993. Int Sym Chitin Enzymol, Senigallia (Italy)).

Another approach has used transgenic expression of recombinant T. harzianum chet42 endochitinase in plants to protect such transgenic plants from Altemaria alternata or Botrytis cinerea infection (Lorito M. et al., 1998. Proc Natl Acad Sci USA. 95:7860) or Venturia inaequalis infection (Bolar J P. et al., 2000. Phytopathology 90:72).

Still another approach has employed transgenic expression of a T. atroviride exochitinase alone or in combination with a T. atroviride endochitinase in plants in an attempt to protect such transgenic plants from V. inaequalis infection (Prakash J P. et al., 2001. Transgenic Research 10:533).

However, all such prior art approaches have failed to provide an adequate solution for providing recombinant fungal chitinases capable of effectively and consistently functioning as anti-fungal agents.

While reducing the present invention to practice, the present inventors have isolated and purified a novel fungal polypeptide exhibiting potent endochitinase activity and activity against chitin-containing organisms. Such a chitinase is therefore highly suitable for the various applications described herein in which chitinases can play unique and useful roles.

Thus, according to one aspect of the present invention, there is provided an isolated chitinase having potent endochitinase activity.

As used herein, the phrase “endochitinase activity” refers to cleavage of the internal β-1,4 glycosidic linkages in chitin molecules to liberate oligomers of at least 3 GluNAc units.

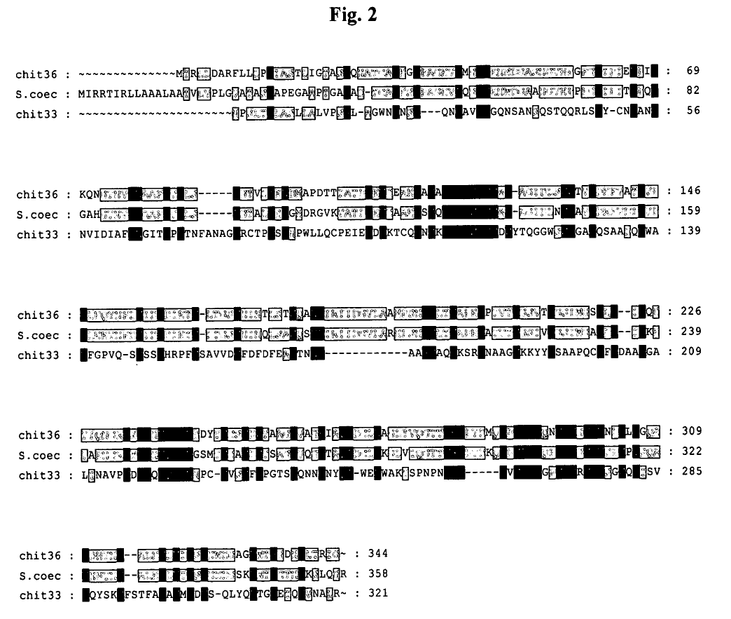

As shown in Example 1 of the Examples section below (FIG. 2), the chitinase of the present invention has the amino acid sequence set forth in SEQ ID NO: 13 and the closest prior art homolog, a protein of Streptomyces coecicolor, only displays 83% similarity with respect to amino acid sequence.

Preferably, the amino acid sequences of the chitinases of the present invention have at least 84% similarity to SEQ ID NO: 13, more preferably at least 90% similarity to SEQ ID NO: 13, more preferably at least 95% similarity to SEQ ID NO: 13, more preferably at least 99% similarity to SEQ ID NO: 13, more preferably 100% similarity to SEQ ID NO: 13 and most preferably 100% identity with SEQ ID NO: 13.

Preferably, percent similarity of amino acid sequences are determined using the Standard protein-protein BLAST [blastp] software (“Positives” output) of the NCBI.

As shown in Example 1 of the Examples section below (FIG. 2), the chitinase of the present invention has the amino acid sequence set forth in SEQ ID NO: 13.

Preferably, the chitinases of the present invention are characterized by an apparent molecular weight being within the range of about 31 to about 35 kDa, as determined via 12% SDS-PAGE following deglycosylation, most preferably about 33 kDa, as determined via 12% SDS-PAGE following deglycosylation.

Preferably, the chitinases of the present invention are characterized by a pI value being within the range of about 4.5 to about 4.9, most preferably by a pI value of about 4.7

Preferably, the chitinases of the present invention are characterized by optimal endochitinase activity at a pH value being within the range of about 4.2 to about 4.8, most preferably at a pH value of about 4.5.

Preferably, the chitinases of the present invention are characterized by optimal endochitinase activity at a temperature being within the range of about 40° C. to about 53° C., most preferably at a temperature of about 48° C.

Preferably, the chitinases of the present invention comprise a signal peptide for extracellular secretion thereof.

Preferably, the signal peptide comprises amino acid residues 1-16 of SEQ ID NO: 13.

While further reducing the present invention to practice, amino acid sequences of a chitinase of the present invention were used to identify and clone a polynucleotide encoding the cDNA of the chitinase.

Thus according to another aspect of the present invention there is provided an isolated polynucleotide encoding a chitinase of the present invention, referred to henceforth herein as “coding polynucleotide”.

As shown in Example 1 of the Examples section which follows, the nucleic acid sequence of the coding polynucleotide of the present invention is set forth in SEQ ID NO: 12. The closest homolog to SEQ ID NO: 12 was found to be the putative chitinase CAB69724 of the fungus Streptomyces coecicolor, with 83% nucleotide sequence identity.

Preferably the coding polynucleotide has a nucleic acid sequence having at least about 84% identity with nucleotides 1-1138 of SEQ ID NO: 12, at least about 84% identity with nucleotides 104-1345 of SEQ ID NO: 12, at least about 84% identity with nucleotides 104-1138 of SEQ ID NO: 12, at least about 84% identity with nucleotides 152-1345 of SEQ ID NO: 12, at least about 84% identity with nucleotides 152-1138 of SEQ ID NO: 12, or more preferably at least about 84% identity with SEQ ID NO: 12; more preferably the coding polynucleotide has a nucleic acid sequence having at least about 90% identity with nucleotides 1-1138 of SEQ ID NO: 12, at least about 90% identity with nucleotides 104-1345 of SEQ ID NO: 12, at least about 90% identity with nucleotides 104-1138 of SEQ ID NO: 12, at least about 90% identity with nucleotides 152-1345 of SEQ ID NO: 12, at least about 90% identity with nucleotides 152-1138 of SEQ ID NO: 12, or more preferably at least about 90% identity with SEQ ID NO: 12; more preferably the coding polynucleotide has a nucleic acid sequence having at least about 95% identity with nucleotides 1-1138 of SEQ ID NO: 12, at least about 95% identity with nucleotides 104-1345 of SEQ ID NO: 12, at least about 95% identity with nucleotides 104-1138 of SEQ ID NO: 12, at least about 95% identity with nucleotides 152-1345 of SEQ ID NO: 12, at least about 95% identity with nucleotides 152-1138 of SEQ ID NO: 12, or more preferably at least about 95% identity with SEQ ID NO: 12; more preferably the coding polynucleotide has a nucleic acid sequence having at least about 99% identity with nucleotides 1-1138 of SEQ ID NO: 12, at least about 99% identity with nucleotides 104-1345 of SEQ ID NO: 12, at least about 99% identity with nucleotides 104-1138 of SEQ ID NO: 12, at least about 99% identity with nucleotides 152-1345 of SEQ ID NO: 12, at least about 99% identity with nucleotides 152-1138 of SEQ ID NO: 12, or more preferably at least about 99% identity with SEQ ID NO: 12; or more preferably the coding polynucleotide is identical to nucleotides 1-1138 of SEQ ID NO: 12, identical to nucleotides 104-1345 of SEQ ID NO: 12, identical to nucleotides 104-1138 of SEQ ID NO: 12, identical to nucleotides 152-1345 of SEQ ID NO: 12, identical to nucleotides 152-1138 of SEQ ID NO: 12, or most preferably identical to SEQ ID NO: 12.

According to the present invention, coding polynucleotides can be genomic polynucleotides, complementary polynucleotides or composite polynucleotides.

As used herein, a “complementary polynucleotide” is a polynucleotide having a nucleic acid sequence resulting from reverse transcription of messenger RNA using a reverse transcriptase or any other RNA dependent DNA polymerase. Such sequences can be subsequently amplified in vivo or in vitro using a DNA dependent DNA polymerase.

As used herein, a “genomic polynucleotide” is a polynucleotide derived from a chromosome which thus reflects a contiguous portion of a chromosome.

As used herein, a “composite polynucleotide” is a polynucleotide which is at least partially complementary and at least partially genomic. A composite sequence can include some exonic sequences required to encode the polypeptide of the present invention, as well as some intronicsequences interposed between the exonic sequences. The intronic sequences can be of any source and typically include conserved splicing signal sequences. Such intronic sequences may further include cis acting expression regulatory elements.

Preferably, the coding polynucleotides of the present invention are complementary DNAs (cDNAs).

The coding polynucleotides of the present invention are capable of genetically directing the production of the chitinases of the present invention, and as such can be used to express high levels of the chitinases of the present invention, for example in in a variety of single cell or multicell expression systems.

Preferably, the coding polynucleotides of the present invention are used to express the chitinases of the present invention in T. harzianum cells, more preferably P. pastoris cells.

For example, as shown in Example 2 of the Examples section which follows, high levels of purified chitinases of the present invention can be obtained by expressing a coding polynucleotide of the present invention in P. pastoris cells under the control of suitable regulatory sequences.

Insertion and/or expression of the coding polynucleotide of the present invention within a host cell is preferably effected by cloning thes polynucleotide within a suitable nucleic acid construct.

Thus, according to the present invention, there is provided a nucleic acid construct comprising the coding polynucleotide of the present invention.

The nucleic acid construct of the present invention can be used to genetically transform a host cell with such a nucleic acid construct.

Thus, according to the present invention, there is provided a host cell comprising the nucleic acid construct of the present invention.

Thus, the chitinases of the present invention, whether isolated from cells or secretions of cells, or generated using the polynucleotide sequences described hereinabove, can be used to form enzymatic compositions enabling application of the enzymatic activity of the chitinases of the present invention towards numerous purposes.

Preferably, the enzymatic compositions of the present invention are composed so as to enable optimal enzymatic activity of the chitinase, according to the physico-chemical parameters described hereinabove.

Preferably, the enzymatic compositions of the present invention comprise the exochitinase EXC-1.

Preferably, the enzymatic compositions of the present invention contain effective concentrations of the chitinases of the present invention to effect the purposes for which they are formulated.

Ample guidance as to such effective concentrations is provided in the Examples section which follows, and in the literature of the art.

As shown in the Example 2 of the Examples section which follows, enzymatic compositions comprising both a chitinase of the present invention and EXC-1 display significantly synergistic and enhanced chitinase activity and activity against chitin-containing organisms such as phytopathogenic fungi, relative to compositions only comprising a chitinase of the present invention.

According to one embodiment of the present invention, the enzymatic composition of the present invention can be formulated as agronomic compositions to protect plants from infection with a chitin-containing pathogen or to treat plants infected with a chitin-containing pathogen.

Preferably, the agronomic composition further comprises an agronomically acceptable carrier or diluent.

An agronomically acceptable carrier can be a solid or a liquid, preferably a liquid, more preferably water. While not required, the agronomic composition of the invention may also contain other additives such as fertilizers, inert formulation aids, i.e. surfactants, emulsifiers, defoamers, dyes, extenders and the like. Reviews describing methods of preparation and application of agronomic compositions are widely available. See, for example, Couch and Ignoffo (1981) in Microbial Control of Pests and Plant Disease 1970-1980, Burges (ed.), chapter 34, pp. 621-634; Corke and Rishbeth, ibid, chapter 39, pp. 717-732; Brockwell (1980) in Methods for Evaluating Nitrogen Fixation, Bergersen (ed.) pp. 417-488; Burton (1982) in Biological Nitrogen Fixation Technology for Tropical Agriculture, Graham and Harris (eds.) pp. 105-114; and Roughley (1982) ibid, pp. 115-127; The Biology of Baculoviruses, Vol. 11, supra, and references cited in the above. Wettable powder compositions incorporating baculoviruses for use in insect control are described in EP 697,170.

According to another embodiment of the present invention, the enzymatic composition of the present invention can be formulated as pharmaceutical compositions which can be used to treat human pathogen infections, such as, for example, Candida albicans.

As used herein a “pharmaceutical composition” refers to a composition of one or more of the active ingredients described hereinabove, or physiologically acceptable salts or prodrugs thereof, with other chemical components such as physiologically suitable carriers and excipients. The purpose of a pharmaceutical composition is to facilitate administration of a compound to an organism.

Hereinafter, the phrases “pharmaceutically acceptable carrier” and “physiologically acceptable carrier” are used interchangeably to refer to a carrier or a diluent that does not cause significant irritation to a treated individual and does not abrogate the biological activity and properties of the active ingredient.

Herein the term “excipient” refers to an inert substance added to a pharmaceutical composition to further facilitate administration of active ingredients. Examples, without limitation, of excipients include calcium carbonate, calcium phosphate, various sugars and types of starch, cellulose derivatives, gelatin, vegetable oils and polyethylene glycols.

Techniques for formulation and administration of the pharmaceutical compositions of the present invention may be found in “Remington's Pharmaceutical Sciences,” Mack Publishing Co., Easton, Pa., latest edition, which is incorporated herein by reference.

Suitable routes of administration may, for example, include oral, rectal, transmucosal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer a pharmaceutical composition in a local rather than systemic manner, for example, via injection of the composition directly into the area of infection often in a depot or slow release formulation, such as described below.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredient into compositions which, can be used pharmaceutically. Proper formulation is dependent upon the route of administration chosen.

Preferably, the pharmaceutical composition is formulated for topical application for treatment of diseases or disorders of the skin or mucosa.

For injection, the active ingredients of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the pharmaceutical composition can be formulated by combining the active agents with pharmaceutically acceptable carriers well known in the art. Such carriers enable the pharmaceutical composition used by the method of the invention to be formulated as tablets, pills, dragees, capsules, liquids, gels, syrups, slurries, suspensions, and the like, for oral ingestion by a patient. Pharmacological compositions for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose compositions such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the agents for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of. e.g. gelatin for use in an inhaler or insufflator may be formulated containing a powder mix of the active ingredient and a suitable powder base such as lactose or starch.

Ophthalmic formulations, eye ointments, powders, solutions and the like, are also contemplated as being within the scope of this invention.

The compositions described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active ingredient in water-soluble form. Additionally, suspensions of the active ingredient may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or formulations, which increase the solubility of the active ingredient to allow for the composition of highly concentrated solutions.

Alternatively, the active ingredient may be in powder form for constitution with a suitable vehicle, e.g., sterile, pyrogen-free water, before use.

The composition of the present invention may also be formulated in rectal compositions such as suppositories or retention enemas, using, e.g., conventional suppository bases such as cocoa butter or other glycerides.

In addition to the formulations described previously, a composition of the present invention may also be formulated for local administration, such as a depot composition. Such long acting formulations may be administered by implantation (for example subcutaneously or intramuscularly) or by intramuscular injection. Thus, for example, the composition may be formulated with suitable polymeric or hydrophobic materials (for example, as an emulsion in an acceptable oil) or ion exchange resins, or as sparingly soluble derivatives such as sparingly soluble salts. Formulations for topical administration may include, but are not limited to, lotions, suspensions, ointments gels, creams, drops, liquids, sprays emulsions and powders.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of active ingredient effective to prevent, alleviate or ameliorate symptoms of disease or prolong the survival of the subject being treated.

Determination of a therapeutically effective amount is well within the capability of those skilled in the art, especially in light of the detailed examples provided herein (refer to the Example 2 of the Examples section which follows).

The therapeutically effective amount or dose can be estimated initially from cell culture assays and cell-free assays (refer to Example 2 of the Examples section below).

Since the enzymatic compositions of the present invention exhibit high anti-fungal activity (refer to Example 2 of the Examples section below) low concentrations thereof can be used in treatment of various fungal diseases, thereby avoiding cytotoxicity.

Regardless, toxicity and therapeutic efficacy of the pharmaceutical compositions described herein can be determined by standard pharmaceutical procedures in experimental animals, e.g., by determining the IC₅₀ and the LD₅₀ (lethal dose causing death in 50% of the tested animals) for a subject ingredient. The data obtained from assays can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma levels of the active ingredient, which are sufficient to maintain the required effects, termed the minimal effective concentration (MEC). The MEC will vary for each composition, but can be estimated from in vitro data; e.g., the concentration necessary to achieve 50-90% inhibition (refer to Example 2 of the Examples section below). Dosages necessary to achieve the MEC will depend on individual characteristics and route of administration. HPLC assays or bioassays can be used to determine plasma concentrations.

Dosage intervals can also be determined using the MEC value. Compositions should be administered using a regimen, which maintains plasma levels above the MEC for 10-90% of the time, preferable between 30-90% and most preferably 50-90%.

It is noted that, in the case of local administration or selective uptake, the effective local concentration of the drug may not be related to plasma concentration. In such cases, other procedures known in the art can be employed to determine the effective local concentration.

Depending on the severity and responsiveness of the infection to be treated, dosing can also be a single administration of a slow release composition, with course of treatment lasting from several days to several weeks or until cure is effected or diminution of the infection state is achieved.

The amount of a composition to be administered will, of course, be dependent on the subject being treated, the severity of the infection, the manner of administration, the judgment of the prescribing physician, etc.

Compositions of the present invention can be packaged in a dispenser device, as one or more unit dosage forms as part of an FDA approved kit, which preferably includes instruction for use, dosages and counter indications. The kit can include, for example, metal or plastic foil, such as a blister pack suitable for containing pills or tablets, or a dispenser device suitable for use as an inhaler. The kit may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising an active ingredient suitable for use with the present invention may also be prepared, placed in an appropriate container, and labeled for treatment of an indicated disease or condition.

Infestations of chitin-containing organisms, such as, for example, insects, routinely cause enormous damage to agricultural crops. Other infestations, such as those in human habitations caused, for example, by cockroaches, ants, and termites, are undesirable and/or cause structural damage to buildings, in the case of termites. Thus, methods of disinfesting chitin-containing pathogens, such as the aforementioned ones, are highly desirable.

According to still another embodiment of the present invention, the enzymatic composition of the present invention can be formulated to combat such infestations of chitin containing organisms.

Preferably compositions for disinfesting chitin-containing organisms comprise suitable carriers or diluents, well known to those of ordinary skill in the art.

As shown in Example 2 of the Examples section below, the chitinases of the present invention exhibit high endochitinase activity and activity against chitin-containing pathogens and, as such, can be used in any application requiring degradation of chitin or activity against chitin containing pathogens.

Preferably, the activate of the chitinase against chitin-containing organisms is inhibition of reproduction thereof, more preferably inhibition of growth thereof and most preferably killing thereof.

Thus, according to still another aspect of the present invention there is provided a method of preventing or treating a disease or a condition associated with or caused by a chitin-containing organism in an individual.

Examples of infections caused by or associated with chitin-containing organisms include, but not limited to, fungal infections, such as cutaneous mycoses subcutaneous mycoses pulmonary mycoses, and candidiasis; protozoal infections, such as toxoplasmosis, malaria (Plasmodium species), leishmaniasis (Leishmania species), Chagas' disease, sleeping sickness (Trypanosoma species), and helminthic infections, such as, nematode infections, schistosomiasis, trichinosis, filariasis and ochocerciasis.

The method is effected by administering to the individual a therapeutically effective amount of the pharmaceutical composition described hereinabove.

According to a preferred embodiment, the method is used to prevent or treat a dermal or mucosal disease or disorder using topical administration of the pharmaceutical composition which is formulated for topical use as described hereinabove.

According to another embodiment, the method is used to prevent or treat an internal (non-cutaneous or non-mucosal) disease or disorder via enteral or subcutaneous administration of the pharmaceutical composition.

The method of the present invention is highly advantageous for use in treating Candida albicans infections, which to date can only be effectively treated by intravenous administration of amphotericin B, which often results in serious adverse affects; such as hypotension and collapse.

As shown in the Examples section below, the chitinases of the present invention display strong activity against growth and spore germination of the fungal phytopathogens Alternaria alternata, Botrytis cinerea or Fusarium oxysporum.

Thus, according to another aspect of the present invention, there is provided a method of preventing or treating a disease or condition associated with or caused by a chitin-containing organism in a plant. According to one embodiment, the method is effected by contacting the plant with an agronomic composition described hereinabove including as an active ingredient the chitinase.

The method can be used to prevent or treat diseases or conditions associated with or caused by arthropods or protozoans, more preferably helminths, or most preferably fungi.

Examples of fungal phytopathogens include fungal species from a wide variety of genera, including Fusarium, Pythium, Phytophthora, Verticillium, Rhizoctonia, Macrophomina, Thielaviopsis, Sclerotinia and the like. Plant diseases caused by fungi include pre- and post-emergence seedling damping-off, hypocotyl rots, root rots, crown rots, vascular wilts and a variety of other forms of symptom development.

Helminthic pathogens include nematode phytopathogens such as those from the genera Meloidogyne, Heterodera, Ditylenchus, and Pratylenchus. Plant diseases caused by nematodes include, but are not limited to, root galls, root rot, lesions, “stubby” root, stunting, and various other rots and wilts associated with increased infection by pathogenic fungi. Some nematodes, such as Trichodorus, Lonaidorus and Xiphenema, are vectors of known viral diseases in a number of plants including Prunus, grape, tobacco and tomato.

Preferably the method is used to prevent or treat diseases or conditions associated with or caused by the fungi Alternaria alternata, Botrytis cinerea or Fusarium oxysporum.

Preferred methods of applying the agronomic compositions of the present invention are leaf application, seed coating and soil application, as disclosed in U.S. Pat. No. 5,039,523.

According to another embodiment, the method of preventing or treating a disease or condition associated with a chitin-containing organism in a plant is effected by expressing the chitinase of the present invention within the plant.

According to a preferred embodiment of the present invention, the step of expressing the chitnase of the present invention in a plant is effected by transforming at least a portion of the cells of the plant with an expression cassette including the chitinase coding sequence (e.g., SEQ ID NO: 12) positioned under the transcriptional control of a plant functional promoter.

The plant functional promoter can be, for example, a constitutive promoter, such as for example, the Cauliflower Mosaic virus (CaMV) 35S promoter or the Ubiquitin promoter; an inducible promoter such as the tetracycline inducible promoter; or a developmentally regulated or tissue specific promoter.

Specific examples of suitable expression cassettes and expression construct harboring such cassettes are given in the Examples section, which follows.

Plant transformation using the expression cassette described above can be effected via any method known in the art for introducing nucleic acid constructs into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276). Such methods rely on either stable integration of the nucleic acid construct or a portion thereof into the genome of the plant, or on transient expression of the nucleic acid construct in which case these sequences are not inherited by a progeny of the plant.

There are two principle methods of effecting stable genomic, integration of exogenous sequences such as those included within the nucleic acid constructs of the present invention into plant genomes:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al. in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

Following transformation plant propagation is exercised. Regeneration can be effected by seed propagation or vegetative propagation methods, which are well known in the art.

In addition to stable genomic expression, the chitinase expression cassette can also be transiently expressed in a whole plant or in specific tissue regions thereof, including, for example, the shoot apical meristem (SAM) or leaves. Thus, in this case, transient transformation methods are utilized for transiently expressing the chitinase of the present invention. Such methods include, but are not limited to, microinjection and bombardment-as described above but under conditions which favor transient expression.

In addition, packaged or unpackaged recombinant virus vector including the chitinase expression cassette can be utilized to infect plant tissues or cells such that a propagating recombinant virus established therein expresses chtinase either in a tissue restricted manner or in the entire plant (systemic infection).

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

As previously discussed, the chitinases of the present invention exhibit potent activity against chitin-containing organisms, and can thus be used to combat infestations of chitin containing organisms.

Preferably, the chitin-containing organism is a helminths or a protozoan, more preferably an arthropods, and most preferably a fungus.

Examples of arthropods include insects, such as ants, wasps, termites, cockroaches, or locust; fleas, ticks, spiders, scorpions, etc.

Chitinases are known to degrade chitin into soluble sugar units, such as N-acetyl-glucosamine monomers or small oligomers of same (Roberts et al., 1988. J. Gen. Microbiol. 134:169. Small soluble compounds, in particular sugars, are known to be associated with or causative of protection against chilling or freezing damage (Finkle, B J. et al. (1985) Cryopreservation of Plant Cells and Organs (Chapter 5), Pages 75-113, CRC Press, Inc. Boca Raton, Fla.; Sakai et al., 1968. Cryobiol. 5:160). It is thus believed that cold damage protection can be mediated by the chitinases of the present invention which may degrade plant polysaccharides (e.g., cleavage of β-1,4 glycosidic bonds in the polysaccharide components of the cell wall such as hemicellulose and pectin) to yield increased levels of soluble monomeric or small oligomeric sugars which in turn results in enhanced protection against freezing or chilling damage. Further support for the use of the chitinases of the present invention as plant anti-freeze agents is provided in Yeh S., 2000. Plant Physiol. 124:1251; U.S. Pat. Nos. 6,235,683, 5,776,448, 5,633,450 and 5,554,524).

Thus, according to still further aspects of the present invention, there is provided a method of preventing or reducing susceptibility of a plant to cold damage.

The method of preventing or reducing susceptibility of a plant to cold damage is preferably effected by expressing the chitinase of the present invention within the plant.

Preferably, chitinase expression is as described above, although the use of cold induced promoters are preferably used to drive chitinase expression. Examples of cold induced promoters include, but are not limited to, Brassica napus BN115 promoter (Sangwan V. et al., Plant J. 2001 July;27(1):1-12) and Arabidopsis Lhcb promoter (Capel J. et al., Plant J. 1998 February;13(3):411).

By virtue of the capacity of chitinases to produce sugars, as described above, the chitinases of the present invention can also be used as a fruit sweetener, as shown in Roberts et al., 1988. J. Gen. Microbiol. 134:169.

The method of utilizing the chitinases of the present invention to produce sugars is preferably effected by expressing the chitinase of the present invention within the plant.

The chitinases of the present invention can also be used as components of chitin-based structures, such as, for example, medical implants, of which controlled degradation is desirable. For, example, in the case of medical implants, drugs incorporated in chitin based capsules or “chitosomes” can be controllably released via the chitinase activity provided by the chitinase comprised in the structure of the implant.

The coding polynucleotides of the present invention can be sequenced and such sequences can be used to identify and clone polynucleotides comprising the upstream non-transcribed regions, and thus the promoters, of the genes encoding the chitinases of the present invention.

Thus, according to yet another aspect of the present invention, there is provides an isolated polynucleotide which functions as a chitin inducible promoter in a host cell, such as a eukaryote cell. This isolated polynucleotide is referred to hereinunder as “regulatory polynucleotide”.

As shown in Example 3 of the Examples section below, the regulatory polynucleotide of the present invention has the nucleic acid sequence set forth in SEQ ID NO: 14, and is less than 30% identical to any other nucleic acid sequence.

Preferably, the nucleic acid sequence of the regulatory polynucleotide is at least about 30% identical to SEQ ID NO: 14, more preferably at least about 30% identical to nucleotides 1110-1139 of SEQ ID NO: 14; more preferably at least about 40% identical to SEQ ID NO: 14, more preferably at least about 40% identical to nucleotides 1110-1139 of SEQ ID NO: 14; more preferably at least about 50% identical to SEQ ID NO: 14, more preferably at least about 50% identical to nucleotides 1110-1139 of SEQ ID NO: 14; more preferably at least about 60% identical to SEQ ID NO: 14, more preferably at least about 60% identical to nucleotides 1110-1139 of SEQ ID NO: 14; more preferably at least about 70% identical to SEQ ID NO: 14, more preferably at least about 70% identical to nucleotides 1110-1139 of SEQ ID NO: 14; more preferably at least about 80% identical to SEQ ID NO: 14, more preferably at least about 80% identical to nucleotides 1110-1139 of SEQ ID NO: 14; more preferably at least about 90% identical to SEQ ID NO: 14, more preferably at least about 90% identical to nucleotides 1110-1139 of SEQ ID NO: 14; more preferably at least about 95% identical to SEQ ID NO: 14, more preferably at least about 95% identical to nucleotides 1110-1139 of SEQ ID NO: 14; more preferably at least about 99% identical to SEQ ID NO: 14, more preferably at least about 99% identical to nucleotides 1110-1139 of SEQ ID NO: 14; or more preferably identical to SEQ ID NO: 14, most preferably identical to nucleotides 1110-1139 of SEQ ID NO: 14.

Preferably, percent similarity of nucleic acid sequences is determined using the Standard nucleotide-nucleotide BLAST [blastn] software (“Identities” output) of the NCBI.

Preferably, the nucleic acid sequence of the regulatory polynucleotide is a genomic DNA sequence.

The regulatory polynucleotides of the present invention possess the capacity to induce specific changes in the expression levels of reporter genes under their regulatory control in cells in response to exposure of such cells to specific environmental conditions, such as stress conditions.

As used herein, a “reporter gene” refers to any expressed polynucleotide positioned downstream of a promoter, so as to be under the regulatory control thereof.

Thus, the regulatory polynucleotides of the present invention can be used to specifically express genes conferring resistance to damage caused by stresses in cells exposed to such stresses, thereby protecting such cells from such damage.

As described above, and as shown in Example 3 of the Examples section below, the regulatory polynucleotides of the present invention have the capacity to induce specific changes in expression levels of reporter genes under the regulatory control thereof in cells in response to exposure of such cells to specific environmental conditions.

Thus, according to yet further aspects of the present invention, there is provided a method of inducing specific changes in expression levels of reporter genes placed under the regulatory control of the promoter containing polynucleotides of the present invention in cells in response to exposure of such cells to specific environmental conditions.

The method is effected by expressing reporter genes under the regulatory control of regulatory polynucleotides of the present invention inserted within the cells.

Recombinant techniques for inserting and expressing coding polynucleotides under the regulate control of regulatory polynucleotides in various cells, such as E. coli, F. oxysporum, yeast, or insect cells, are widely practiced and well known to ordinary practitioners in the art.

According to one embodiment, the exogenous regulatory polynucleotide is used to regulate expression of reporter genes being exogenous to the cells. This enables expression of essentially any desired gene in the cell under the regulatory control of the exogenous regulatory polynucleotide.

According to another embodiment, the exogenous regulatory polynucleotide is used to regulate expression of reporter genes being endogenous to the cells. This enables expression of essentially any desired endogenous gene in the cell under the regulatory control of the exogenous regulatory polynucleotide.

Polynucleotides such as the regulatory polynucleotides of the present invention can be inserted upstream of coding sequences in a genome, so as to place such coding sequences under the regulatory control thereof, by using techniques, such as “promoter knock-in” techniques, well known to those of ordinary skill in the art.

Preferably, the cells in which exogenous, regulatory polynucleotides are used to regulate expression of reporter genes are Trichoderma harzianum cells.

As shown in Example 3 of the Examples section below, the regulatory polynucleotides of the present invention induce changes in expression levels of gfp or chit36 reporter genes in T. harzianum cells in response to specific environmental conditions, such as stress conditions.

The inducible expression of stress-protective polypeptides by plant cells in response to specific stress conditions can be used to protect plants from such stress conditions.

Thus, according yet still another aspect of the present invention, there is provided a method of reducing the susceptibility of plants to damage resulting from exposure to specific environmental conditions.

The method is effected by expressing in plants, plant tissues or plant seeds, gene products of reporter genes capable of reducing the susceptibility of the plants, or such parts thereof, to such damage. Such reporter genes are expressed under the regulatory control of the regulatory polynucleotides of the present invention inserted into the cells of such plants, plant tissues or plant seeds.

Thus, according to another aspect of the present invention, there are provided plants, plant tissues or plant seeds comprising the coding polynucleotides of the present invention.

Plants, plant tissues or plant seeds expressing the chitinases of the present invention may be used to evaluate pathogen resistance, in particular resistance to fungal pathogens. Fungal pathogen resistant lines may be used in breeding programs to yield commercial varieties with enhanced fungal pathogen resistance. The advantages of the plants, or parts thereof, produced according to the present invention are a reduced need for fungicide treatment, lowering costs of material, labor, and environmental pollution, or prolonged shelf-life of products (e.g. fruit, seed, and the like). Furthermore, post-harvest losses may be reduced due to the presence of the chitinases expressed by harvested plants or plant tissues.

The methods of using the regulatory polynucleotides of the present invention to regulate expression of reporter genes are preferably used to induce decreases in reporter gene expression levels in response to elevated glucose concentrations, most preferably to induce increases in reporter gene expression levels in response to stress conditions.

Methods of using the exogenous regulatory polynucleotides of the present invention to regulate expression of reporter genes are preferably used to increase reporter gene expression levels in response to stress conditions such as osmotic stress or nitrogen starvation, more preferably in response to exposure to a chitin-containing organism, a temperature extreme, or an elevated chitin concentration.

According to one embodiment, methods of using the exogenous regulatory polynucleotides of the present invention to regulate expression of reporter genes are used to increase reporter gene expression levels in response to temperatures being no greater than about 4° C., more preferably no greater than about 0° C., or no lower than about 40° C.

According to another embodiment, methods of using the exogenous regulatory polynucleotides of the present invention to regulate expression of reporter genes are used to increase reporter gene expression levels in response to glucose concentrations being no lower than about 50 g/L.

According to a preferred embodiment, methods of using the exogenous regulatory polynucleotides of the present invention to regulate expression of reporter genes are used to increase reporter gene expression levels in response to chitin concentrations being no lower than about 2 g/L.

As shown in Example 3 of the Examples section below, T. harzianum cells expressing a chitinase of the present invention, under the regulatory control of a regulatory polynucleotide of the present invention exhibit decreased reporter gene expression levels in response to elevated glucose concentrations and increased reporter gene expression levels in response to all of the aforementioned stress conditions.

Thus, the regulatory polynucleotides of the present invention are highly suitable for protecting plants from stress, for example by using as a reporter gene the chitinase of the present invention which, being a chitinase, has proven capacities to protect cells against cold damage and chitin-containing organisms, such as phytopathogenic fungi.

Alternately, the reporter gene can be a heat-shock protein, such proteins having been widely demonstrated to protect cells from stresses.

As described above the nucleic acid constructs of the present invention are expressed in cells, such as plant cells. Methods of expressing nucleic acid constructs in plant cells are well known to one of ordinary skill in the art.

Numerous plant functional expression promoters and enhancers which can be either tissue specific, developmentally specific, constitutive or inducible can be utilized by the constructs of the present invention, some examples are provided hereinunder.

Nucleic acid constructs for expressing the coding polynucleotide in plant cells must include suitable regulatory sequences.

Such regulatory sequences include promoters which can direct gene expression in plant cells (including DNA containing organelles). Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric, i.e., formed of portions of at least two different promoters.

Thus, the plant promoter employed can be a constitutive promoter, a tissue specific promoter, an inducible promoter or a chimeric promoter.

Examples of constitutive plant promoters include, without being limited to, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter.

Examples of tissue specific promoters include, without being limited to, bean phaseolin storage protein promoter, DLEC promoter, PHS* promoter, zein storage protein promoter, conglutin-γpromoter from soybean, AT2S1 gene promoter, ACT11 actin promoter from Arabidopsis, napA promoter from Brassica napus and potato patatin gene promoter.

The inducible promoter is a promoter induced by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr203J and str246C active in pathogenic stress.

The construct according to the present invention preferably further includes an appropriate and unique selectable marker, such as, for example, an antibiotic resistance gene.

The constructs according to the present invention can be shuttle vectors which can propagate both in E. coli (wherein the construct comprises an appropriate selectable marker and origin of replication) and be compatible for propagation in cells, or integration in the genome, of a plant.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular; biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8^{th} Edition), Appleton & Lange, Norvalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Potent Antifungal Activity of the Novel Trichoderma harzianum Endochitinase CHIT36

A very broad range of fungal, nematode and insect phytopathogens are responsible large agricultural losses. Similarly, in humans and animals, fungal, protozoan and parasitic pathogens, are responsible for causing numerous diseases of major clinical impact. Since such pathogens are chitin-containing, since disruption of the chitin-containing structures thereof is highly harmful to such organisms, and since plants and vertebrates do not express chitin, chitin can serve as a selective target for anti-pathogenic agents. In order to provide such an anti-pathogenic agent, the present inventors have cloned and isolated a novel and potent fungal chitinase, as follows.

Materials and Methods:

Cloning of chet36 cDNA: T. harzianum chromosomal DNA was isolated as previously described (Raeder U. and Broda P., 1985. Lett. Appl. Microbiol. 1:17). Total RNA was isolated with the EZ-RNA kit (Biological Industries, Israel) and mRNA was purified with the PolyATrac kit (Promega). Cloning of chit36 cDNA was performed via 5′ and 3′ RACE PCR using the SMART RACE cDNA Amplification Kit (Clontech). All other nucleic acid manipulations were carried out as previously described (Sambrook J. et al., 1989. “Molecular cloning: a laboratory manual”, 2^{nd} ed., vol. 1. Cold Spring Harbor Laboratory Press, Cold Spring Harbor, N.Y.).

Generation of CHET36 expression vector: Vector pAN7 encoding the E. coli hph hygromycin resistance was purchased from Stratagene. For generation of T. harzianum transformants constitutively expressing CHIT36, the chit36 open reading frame (ORF) was inserted in the XbaI/NsiI sites of expression vector pRLMex-30 (kindly provided by Prof. R. L. Mach) downstream of the pki1 promoter to generate vector pRL-36.

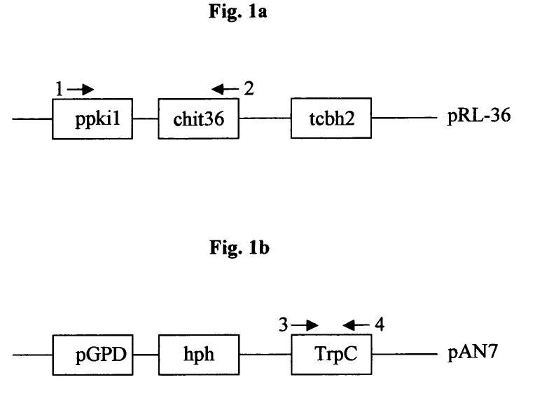

Transformation procedure and selection: Microprojectile bombardment of intact T. harzianum conidia was performed essentially as described in (Hazell B W. et al., 2000. Letters in Appl. Microbiol. 30:282), with minor modifications. Briefly, 0.4 μm SE tungsten particles loaded with a total of 1 μg DNA were used for co-transformation with plasmids pAN7 and pRL-36. Co-transformants were screened for expression of the selectable marker by plating on PDA supplemented with 300 μg/ml hygromycin B. Colonies were grown on SM minimal medium supplemented with 5% glucose and after two days culture filtrates were tested for endochitinase activity. Single spores were isolated from selected colonies and spread on selective medium. This process was repeated twice. Transformants were also screened by PCR for transformation by both plasmids using the primer pairs: 5′-CGTGGCAGCTCGAGATAACG-3′ (SEQ ID NO: 1; corresponding to nucleotide numbers −744 to −725 of the pki1 promoter in pRLMex-36) and 5′-CGGTGCCATAGTCAAGCCAAA-3′ (SEQ ID NO: 2; corresponding to the sequence complementary to that encoding peptide FGLTMAP (SEQ ID NO: 5) of chit36) for amplification of a pRL-36 specific sequence (FIG. 1a); and 5′-AGGTACCGATTTAATAGCTCCATGTCAAC-3′ (SEQ ID NO: 3; corresponding to the 5′ end of trpc in pAN7) and 5′-AGGTACCGTCTAGAAAGAAGGATTACCTC-3′ (SEQ ID NO: 4; corresponding to the 3′ end of trpc in pAN7) for amplification of a pAN7 specific sequence (FIG. 1b).

Strains, media and culture conditions: Trichoderma harzianum Rifai, strain TM, was grown on potato dextrose agar (PDA). For recovery of chitinolytic activity, T. harzianum was grown on SM medium (Okon, Y. et al., 1973. J. Gen. Microbiol. 74:251) with 0.2% (w/v) colloidal chitin as the sole carbon source, so as to upregulate endogenous chitinase expression, or with 5% glucose, so as to downregulate endogenous chitinase expression.

Protein sequence and oligonucleotides: Gel slices containing purified CHIT36 (Haran S H. et al., 1996. Phytopat. 86:980) were analyzed via Procise Protein Sequencer (Perkin-Elmer ABD 492; Bletterman Macromolecular Res. Laboratory, Hebrew University, Faculty of Medicine, Jerusalem). The internal CHIT36 peptides GYWENWD (SEQ ID NO: 6) and YDMQVPG (SEQ ID NO: 7) were amino acid-sequenced and the sequences were used to design the degenerate primers GG(N)TA(C/T)TGGGA(A/G)AA(C/T)TGGGA (SEQ ID NO: 8) and CC(N)GG(N)AC(T/C)TGCAT(G/A)TC(G/A)TA (SEQ ID NO: 9), respectively.

Isolation of extracellular proteins and Western blots: Two day-old cultures of T. harzianum were filtered and the culture filtrates were concentrated 40-fold using Vivaspin concentrators with a 10 kDa cut-off membrane (Vivascience). Western blots were performed according to standard procedures in 12% polyacrylamide gels. Anti-CHIT36 polyclonal antibody (Haran S H. et al., 1996. Phytopat. 86:980) was used at 1:1000 dilution for detection of CHET36. Protein concentrations were determined via Bradford assay (Bio-Rad)

Biological Assays:

Chitinase activity assays: Crude chitinolytic activity in pathogen confrontation assays was determined as previously described (Inbar J. and Chet. I., 1991. Soil Biol. Biochem. 23:973). Briefly, 50 μl aliquots of culture filtrates of transformants grown in glucose rich medium were assayed with the fluorescent substrate 4-methylumbelliferyl-N,N′-β-D-N,N′-diacetylchitobioside (4-MU-(GlcNAc)₂. Assays of enzymatic activities on SDS-PAGE gels were performed according to (Haran S. et al., 1995. Mycol. Res. 99:441).

For specific chitinase activity assays of culture filtrates, strains were cultured in SM medium containing 5% glucose for 48 h, the mycelia were collected and the dry weight recorded for the calculation of the specific chitinase activity. Chitinase activity in 440 μl culture filtrate aliquots were determined via a fluorescence assay utilizing pNp-N-acetylglucosamine (pNp-NAGA), as previously described (Inbar. J. and I. Chet. 1991. Soil Biol. Biochem. 23:973).

Conidia germination assay: Since there is little protein apart from CHIT36 in culture filtrate of chit36 transformants grown in medium supplemented with 5% glucose, (data not shown), culture filtrate of tranformants was used directly for analyzing the biological effect of CHIT36, as compared to filtrate of WT culture in a state of carbon repression, in which no chitinolytic activity was detectable. Conidia germination bioassays were performed essentially as previously described (Lorito M. et al., 1996. MPMI 9:206). Briefly, 60 μl aliquots of assay mixture containing 2,000-3,000 Botritys cinerea or T. harzianum conidia in PDB were incubated in a flat-bottomed ELISA plates at 22° C. Culture filtrates from transformant or wild-type conidia grown in glucose rich media or 0.2% colloidal chitin were added to the reaction mixture following dilution to final concentration via dialysis against 0.1 M potassium acetate buffer (pH 4.5). Plates were examined after 20 h using an inverted microscope and percentages of germinating spores were determined. The experiment was repeated twice in triplicate.

Phytopathogen growth inhibition assays: Petri dishes with agar SM medium supplemented with 5% glucose were covered with cellophane discs allowing passage of cellular secretions to the agar but not cells (Scoth, Cergy, Pontoise, France) and inoculated with a 5 mm diameter mycelial disc of transformed T. harzianum. After 2 days of incubation at 28° C. the cellophane was removed and the plates were inoculated with a 5 mm diameter mycelial discs of phytopathogenic fungi, as indicated. Colony radii were measured 48 and 96 h following inoculation. Chitinase secretion on replicate plates was checked with the fluorescent substrate 4-MU-(GlcNAc)₂ and visualized under UV light. Non-pre-inoculated plates were used as control for normal growth.

Experimental Results:

Isolation and characterization of the chit36 gene: Q-Sepharose column eluate fractions of 0.2% colloidal chitin induced T. harzianum culture filtrate containing CHIT36 activity (Haran S H. et al., 1996. Phytopat. 86:980) were pooled and separated via SDS-PAGE for sequencing. Since the N-terminus was found to be blocked for sequencing, the protein was digested with trypsin and the amino acid sequences of the internal peptides VLMGYWENWDGASNGVHPGF (SEQ ID NO: 10) and IPYDMQVPGLPAQNG (SEQ ID NO: 11) were determined.

The chit36 ORF was cloned and sequenced (SEQ ID NO: 12), and the complete chit36 cDNA sequence was found to be 1242 bp in length (nucleotides 104-1345 of SEQ ID NO: 12), including a 207 bp 3′ untranslated region. A search for homologs of chit36 cDNA using the NCBI's BLAST software in standard nucleotide-nucleotide [blastn] mode identified the closest homolog as being the putative chitinase CAB69724 of the fungus Streptomyces coecicolor, with 83% nucleotide sequence identity.

The chit36 cDNA was found to encode a 344 amino acid polypeptide (SEQ ID NO: 13) with a 16-residue putative N-terminal signal peptide sequence, identified as previously described (von Heijne, G. 1986. Nucleic Acids Res. 14:4683), with an expected molecular mass of 36 kDa after processing. The protein CHIT36 from T. harzianum TM has previously been shown to be a heat resistant endochitinase with an apparent molecular weight of 33 kD and a pI of 4.8. Expression of chit36 is induced by chitin or via antagonistic interaction with R. solani and is repressed by glucose (Haran S. et al., 1995. Mycol. Res. 99:441; Haran S H. et al., 1996. Phytopat. 86:980). Similar known endochitinases include an endochitinase with a molecular mass of about 37 kDa and similar pI value purified from T. harzianum Rifai CECT 2413 (Limon M C. et al., 1995. Curr. Genet. 28:478), and an endochitinase purified from T. harzianum isolate 1051 (Limon M C. et al., 1999. Phytopat. 89:254).

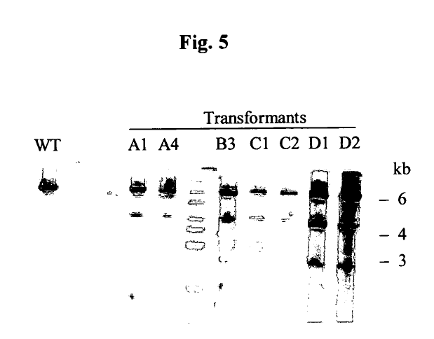

Amino acid sequence alignment searches, performed using the standard protein-protein Blast (blastp) mode of the NCBI's BLAST software package, showed that the amino acid sequence of CHIT36 has 83% similarity to the putative chitinase CAB69724 of the fungus Streptomyces coecicolor, 50% similarity to CHIA of the bacterium B. cereus, and 48% similarity to CHITD of the bacterium B. circulans. No significant similarity was found with other eukaryotic chitinases and, as shown in FIG. 2, no significant homology with the small endochitinase chit33 of T. harzianum (Limon M C. et al., 1995. Curr. Genet. 28:478) was found either. According to a Blast CD-Browser search, CHIT36 belongs to chitinase class II group (family 18) with a typical (β-α) 8-fold barrel. No introns were identified in the coding region in the genomic DNA and Southern blot analysis of genomic DNA cut with EcoRI, which does not cut in the gene sequence, showed that chit36 is a single copy gene (FIG. 5).

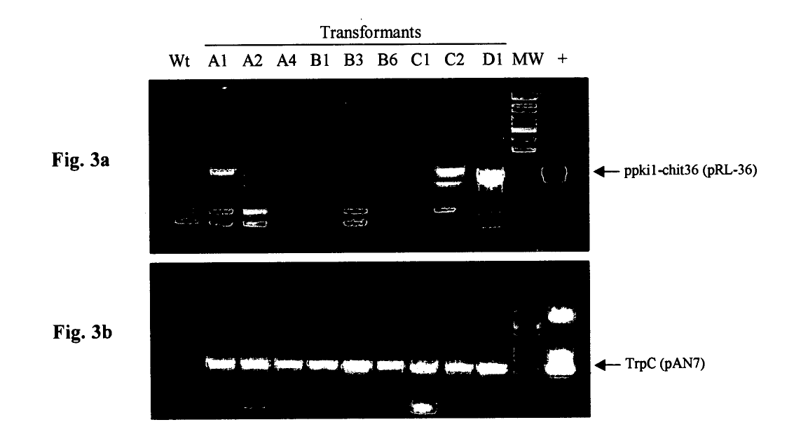

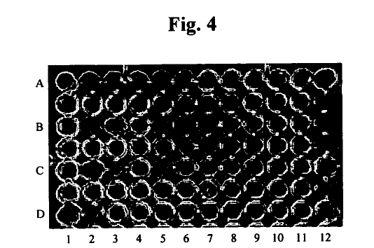

Isolation and characterization of transformats: Whereas transformation frequencies achieved by co-bombardment of pAN7 and pRL-36 were relatively low (1-10 transformants/μg total DNA), the efficency was very high. As shown in FIG. 3a, 7 out of 9 transformants selected on hygB medium were also positive in the PCR screening for the ppki1-chit36 transgene sequences. Transformants whose culture filtrates displayed chitinolytic activity when grown in medium supplemented with 5% glucose (e.g., A1, B3, C2, D1, FIG. 4) were chosen for further characterization. Southern analysis of genomic DNA digested with EcoRI, which cuts only once in pRL-36, revealed that the genomic DNA of stable transformants contained up to two integrations of ppki1-chit36 (FIG. 5). These were in the same locations in different transformants, indicating a preferred recombination site of the transgene. The signal in the D1 and D2 tranformants was found to be more intense, indicating a probable multicopy insertion of the construct. In the case of D1, this correlates with the higher levels of specific chitinase activity in culture filtrate conditioned of this transformant (Table 1). Cultures of chit36 tranformants were found to contain higher levels (up to 40-fold) of specific chitinase activity than the wild-type on grown in glucose rich medium (Table 1). With chitin as carbon source there was no significant difference from the wild-type (data not shown) as was previously reported for other overexpressing mutants (De Marco J L. et al., 2000. World J. of Microbiol and Biotechn. 16:383).

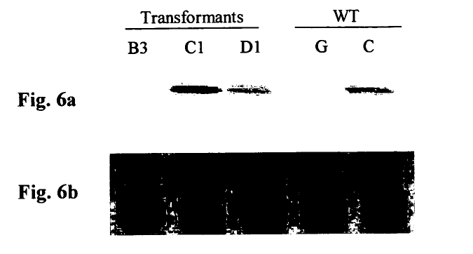

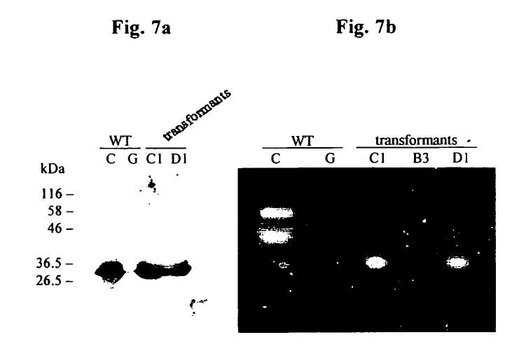

Also correlating with the chitinase activity levels shown in Table 1, transformants C1 and D1, were found to express higher levels of chit36 mRNA than transformant B3 when grown in glucose rich medium (FIGS. 6a-b). Secreted CHIT36 protein was detected in transformants at the expected molecular weight via Western immunoblotting assay (FIG. 7a) and as the only chitinolytic enzyme via in situ gel chitinase activity assays of culture filtrate proteins separated via SDS-PAGE (FIGS. 7a-b).

TABLE 1
Specific chitinase activity in cultures of
T. harzianum CHIT36 transformants

| Treatment | Specific chitinase activity (units/g dry weight)* |
|---|---|
| wild-type | 10 ± 1.7 |
| transformant A1 | 12 ± 4 |
| transformant B3 | 78 ± 22 |
| transformant C1 | 257 ± 35 |
| transformant D1 | 360 ± 45 |

*1 unit of chitinase activity was defined as the amount of enzyme required to increase absorbance at 400 nm by 1 O.D. unit · ml⁻¹ · h⁻¹.

Biological Activity of CHIT36:

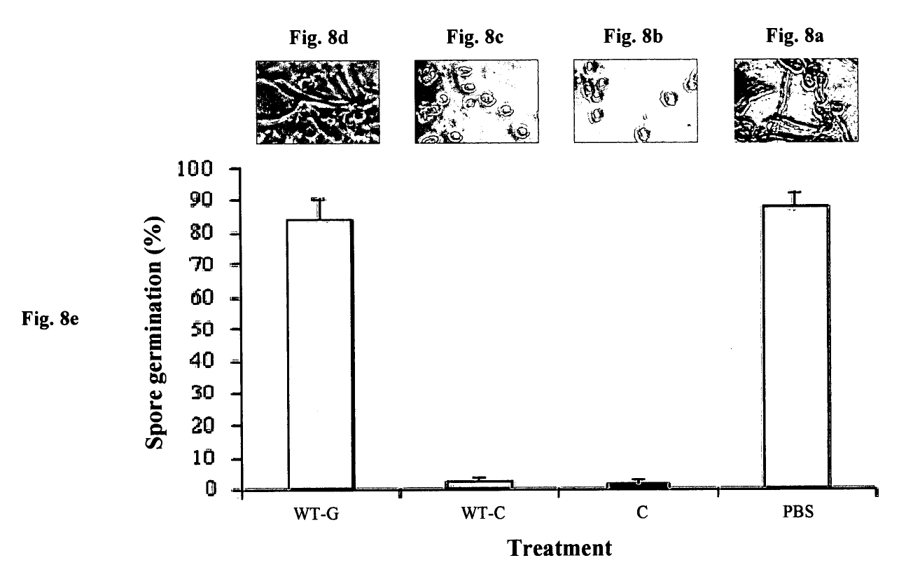

CHIT36 inhibits germination of B. cinerea conidia: After 20 h of incubation in the presence of T. harzianum transformant C culture filtrate, an almost complete inhibition in germination of B. cinerea conidia was observed (FIGS. 8a-e).

Recombinant CHIT36 inhibits growth of fungal phytopathogens: As shown in Table 2, growth of both of the fungal phytopathogens F. oxysporum and S. rolfsii was markedly inhibited by growth on agar plates containing chit36 transformant versus wild-type T. harzianum secretions. This inhibition is most likely due to the constitutive secretion of CHIT36 in the culture plates where the transformants were previously grown.

Chitinase secretion was detectable via fluorescence assays utilizing the substrate 4-MU-(GluNAc)₂ in plates pre-inoculated with transformants but not in wild-type T. harzianum (data not shown).

These results therefore demonstrate that the novel recombinant T. harzianum CHIT36 endochitinase of the present invention has potent endochitinase activity capable of inhibiting growth of various fungal phytopathogens, including, for the first time, growth of F. oxysporum. As such, the rCHIT36 endochitinase of the present invention is superior to all prior art recombinant fungal chitinases as an anti-fungal agent and is therefore highly suitable, for example, for protection of crop plants from fungal pathogens.

TABLE 2
Growth inhibition fungal phytopathogens in agar
plates containing chit36 transformant secretions

| Agar | F. oxysporum | | S. Rolfsii | |
|---|---|---|---|---|
| conditioning | 48 h | 96 h | 48 h | 96 h |
| None | 1.46 ± 0.08 | 2.5 ± 0.0 | 2.5 ± 0.0 | 4.3 ± 0.02 |
| transformant C | 056 ± 0.09 | 1.1 ± 0.14 | 0.44 ± 0.11 | 1.86 ± 0.35 |
| transformant D | 0.52 ± 0.04 | 1.06 ± 0.13 | 0.64 ± 0.11 | 2.56 ± 0.13 |
| WT | 0.9 ± 0.07 | 1.86 ± 0.21 | 1.28 ± 0.17 | 3.5 ± 0.35 |
| T. harzianum | | | | |

Data shown are colony radii (cm) and represent the average of 5 replicates from one representativ experiment out of three.

### Example 2

Combined CHIT36 Endochitinase and EXC1-Y Exochitinase Possess Potent and Synergistic Chitinase and Anti Fungal Activities

Both exochitinases and endochitinases are required for efficient breakdown of chitin, a structural polymer of a large number of plant and vertebrate pathogens or pests, such as fungi, insects, protozoans and parasites. Since the chitin-containing structures of such pathogens or pests are critical for their survival and since chitin is not expressed in plant or vertebrate cells, chitin can serve as a selective target for agents directed against such pathogens.

While reducing the present invention to practice, the present inventors have unexpectedly uncovered that combined CHIT36 endochitinase and EXC1-Y exochitinase display potent and synergistic chitinase activity and anti-chitin-containing pathogen activity, as follows.

Materials and Methods:

Cloning of vector for expression CHIT36 in P. pastoris: The 0.96 kb EcoRI-NotI fragment encoding T. harzianum Rifai TM CHIT36, minus the first 16 signal peptide encoding residues, was fused in frame to the α-factor signal sequence at the EcoRI site of the expression vector pPIC9K (Invitrogen Co. U.S.A.) to generate plasmid pPIC9K-36.

Transformation and selection of P. pastoris: The bacterial and fungal strains E. coli JM109 and P. pastoris GS115 (his4) were used as cloning and expression hosts respectively. Yeast cells were grown in YPD, BMGY, BMMY, MD or MM media prepared as described in “P. pastoris Expression Manual” (Invitrogen Co. U.S.A.). The yeast P. pastoris, constitutes an efficient overexpression system suitable for the production of large amounts of enzymes. Transformations were performed via electroporation of GS 115 competent cells with 10 μg of BglII linearized pPIC9K-36 using the Gene-Pulser Electroporator (BioRad).

Histidine positive transformant colonies were selected on minimal MD medium without histidine supplementation and replicated on inductive MM medium (1% methanol) and grown for 2 days at 30° C. Replica plates were overlayed with 10 ml of 1% agarose in 0.1 M potassium acetate buffer (pH 4.5) containing 1 mg of 4-methylumbelliferyl-N,N′-β-D-N,N′-diacetylchitobioside (4-MU-(GlcNAc)₂) and chitinase secreting colonies were monitored via release of the fluorescent product 4-methylumbelliferone visualized under UV light, as previously described (Draborg H. et al., 1996. Curr. Genet. 29:404).

Recombinant CHIT36 purification: Culture filtrates were dialyzed against 50 mM sodium acetate buffer pH 6.7, dialyzed solutions were applied to Q-Sepharose anion exchange columns (HiTrap Q, Pharmacia) previously equilibrated with the same buffer, and the columns were eluted with a continuous gradient of 0-1 M NaCl in 50 mM sodium acetate buffer pH 6.9 at a flow rate of 60 ml/h.

Determination of isoelectric point (pI) and gel activity staining: The pI of rCHIT36 was determined and in situ gel chitinase activity assays were performed as previously described (Haran S. et al., 1995. Mycol. Res. 99:441).

Enzyme assays: chitinase activity on colloidal chitin was tested as previously described (Miller GL., 1959. Analyt. Chem. 31:426) using N-acetyl-D-glucosamine (GlcNAc) as standard. Generation of monomeric GlcNAc product was measured as previously described (Reissig J L. et al., 1959. J. Biol. Chem. 217:959) using the p-dimethylaminobenzaldehyde (DMAB) reagent. The following chromogenic oligomers of GlcNAc p-nitrophenyl-N-acetyl-β-D-glucosaminide (pNP-GlcNAc) and p-nitrophenyl-β-D-N-N′-diacetylchitobiose [(pNP-GlcNAc)₂] (Inbar J. and Chet I. 1991. Soil Biol. Biochem. 23:973) were used as substrates. One unit of enzyme was defined as the amount of enzyme required to liberate 1 μmol product/min under the described assay conditions. Activities on laminarin or carboxymethylcellulose (CMC) were determined as previously described (Somogyi M., 1952. J. Biol. Chem 195:19).

Anti-fungal activity bioassay and data analysis: Bioassays were performed as previously described (Lorito et al. 1994. Phytopathology 84:398). 60 μl aliquots of assay mixture containing 2,000-3,000 conidia in potato dextrose broth (PDB) were incubated in flat-bottomed ELISA plates at 24° C. Assay plates were examined after 20 h the using an inverted microscope and percentages of germinating spores were determined. All assays included sterile water controls instead of the tested enzymes dissolved in water. Each experiment was repeated twice in triplicate.

The ED₅₀ values of the enzymes tested were calculated by regression analysis using a polynomial regression with R² ranging from 0.93 to 0.98. Dose response curves were determined using 7 enzyme concentrations. The antifungal synergistic effect was also verified according to Richer's formula: (E₀)(Xa+Yb)>(E₀)(X+Y)(A) and (E₀)(X+Y)(B), where E₀ is the percent inhibition, A and B the two components, and X and Y, the concentrations of each component, respectively (Richer D L., 1987. Pestic Sci. 19:309).

Gel in situ chitinase assays and Western immunoblotting assays: P. pastoris chit36 transformants were evaluated for secretion of CHIT36 in liquid culture. Single colonies of positive transformants were grown to saturation in 10 ml BMGY medium for 2 days. Cells were harvested by centrifugation, resuspended in 2 ml of BMMY medium, and after a further 24 h of culture, 20 μl aliquots of culture filtrate were analyzed via native PAGE and SDS-PAGE.

Recombinant CHIT36 purification: performed as described in Example 1, above.

Experimental Results:

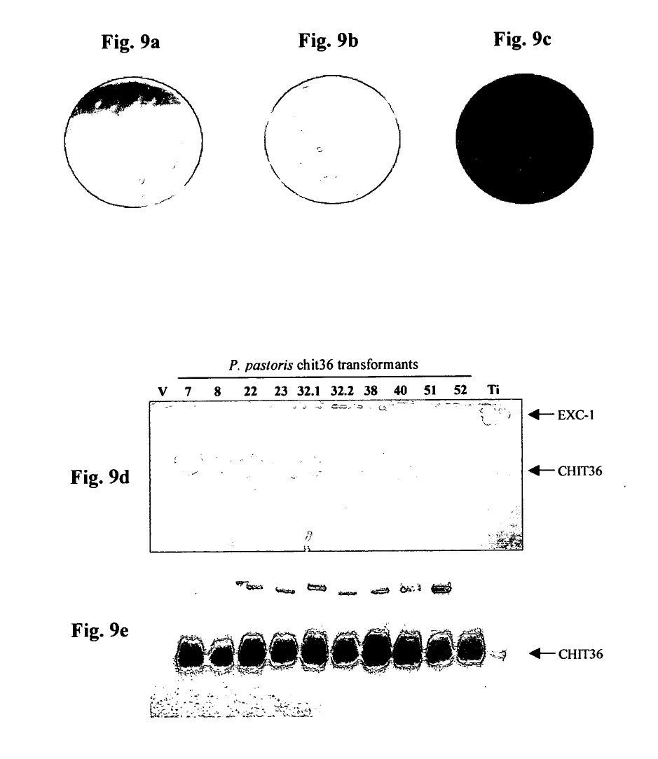

P. pastoris chit36 transformants secrete high levels of functional CHIT36: About 60% of the positive transformant colonies tested were found to be positive for CHIT36 endochitinase secretion (FIGS. 9a-b) relative to mock-transfected colonies (FIG. 9c). Enzymatically active CHIT36 was found to accumulate in the culture filtrate at different levels in the different clones, as determined via gel in situ chitinase assays (FIG. 9d), and Western immunoblotting assays (FIG. 9e) of filtrate proteins separated via native PAGE. Recombinant CHIT36 (FIG. 9e, lanes 2-11) migrated at the same apparent molecular weight of 36.5 kDa as endogenous Trichoderma CHIT36, (FIG. 9e, lane 12).

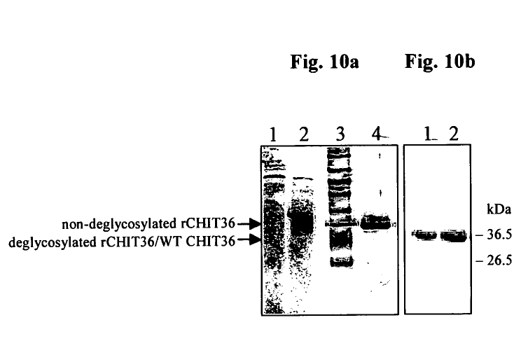

Large-scale recombinant CHIT36 production: One colony expressing high levels of CHIT36 was selected and cultured for CHIT36 production in a 100-fold scaled up final volume of 200 ml. The purified protein (Table 3) was detected as a single band migrating at an apparent molecular weight of 39 kDa via SDS-PAGE (FIG. 10a, lane 4). Whereas rCHIT36 produced by P. pastoris transformants (FIG. 9e, numbered lanes) migrated at the same molecular weight as endogenous T. harzianum CHIT36 (FIG. 9e, Ti lane) in native gels, rCHIT36 (FIG. 10a, lane 4) revealed a higher apparent molecular weight than native CHIT36 (FIG. 10a, lane 2) when analyzed via SDS-PAGE, suggesting that it was produced in a hyperglycosylated form, a known potential characteristic of the Pichia expression system (Gellissen G., 2000. Appl. Microbiol. Biotechnol. 54:741). When deglycosylated via treatment with PNGaseF (New England Biolabs, USA), the apparent molecular weight of rCHIT36 as determined via SDS-PAGE decreased to that of wild-type CHIT36 (FIG. 10b, lanes 1 and 2, respectively), at the expected apparent molecular weight of 36.5 kDa. providing evidence the that amino acid sequence of the recombinant protein was properly synthesized since it migrated at the expected apparent molecular weight in its normal glycosylation state.

TABLE 3
Purification of recombinant CHIT36 from culture filtrate
of P. pastoris chit36 transformants

| | | | | Specific | |
|---|---|---|---|---|---|
| | | Total | Total | chitinase | |
| | Volume | protein | activity | activity | Purification |
| Purification stage | (ml) | (mg) | (units*) | (units/mg) | factor |
| pre-dialysis | 50 | 12.5 | 15 | 1.2 | — |
| post-dialysis | 50 | 12.5 | 15 | 1.2 | — |
| post-Q-Sepharose | 6 | 3.6 | 9.6 | 2.6 | 2.1 |
| column | | | | | |

*1 unit = OD₄₀₀ · 0.5 ml⁻¹ · 15 min⁻¹ using pNP-(GlcNAc)₂ as substrate.

Biological activity of recombinant CHIT36: Hyperglycosylation did not affect the enzymatic activity of rCHIT36. Substrate specificity and enzymatic activity of the recombinant protein were found to be in agreement with the data obtained with the partially purified native form of CHIT36 (Haran et al., 1996, Viterbo et al., 2001). The optimal activity of the enzyme was found to be between 40-53° C. with a peak optimum at 48° C., at pH 4.5, as determined by the use of pNP-(GlcNAc)₂ as substrate, and its pI, as determined via isoelectrofocusing gel analysis, was found to be 4.7. The purified rCHIT36 was capable of hydrolyzing colloidal chitin, as shown by liberation of reducing sugars, and was highly active on pNP-(GlcNAc)₂ substrate (Table 4).

TABLE 4
Substrate specificity of recombinant CHIT36

| Substrate | Specific chitinase activity (units*) |
|---|---|
| colloidal chitin | 1.25 |
| PNP-(GlcNAc)₂ | 200 |

*1 unit = amount of enzyme able to liberate 1 μmolproduct/min

CHIT36 and EXC1-Y potently and synergistically inhibit spore germination of fungal phytopathogens: Spore germination of the three plant pathogens B. cinerea, A. alternata and F. oxysporum v. melonis was significantly inhibited by rCHIT36 (Table 5), with a 50% effective dose (ED₅₀) of 43-83 μg/ml, a range similar to that reported for other chitinases from T. harzianum in similar experiments (Lorito et al., 1993. Phytopathology 83:302; Lorito, M. et al., 1994. Phytopathology 84:398). The ED₅₀ for EXC1-Y was found to be 90-151 μg/ml. When 10 μg/ml EXC1-Y was added to increasing concentrations of CHIT36 the ED₅₀ value of the combined enzymes was 16.6 and 17.8 μg/ml for A. alternata and B. cinerea, respectively, and 49 μg/ml for F. oxysporum (Table 5). According to Richer's formula the E₀ values for the combination of the enzymes were higher than for each individual enzyme (data not shown).

TABLE 5
Synergistic inhibition of spore germination of
fungal phytopathogens by rCHIT36 and EXC1-Y.

| | | Fusarium | Botrytis |
|---|---|---|---|
| | Alternaria alternata | oxysporum | cinerea |
| CHIT36 | 43.4 | 83 | 55.3 |
| EXC1-Y | 90 | 112.9 | 151 |
| CHIT36 + EXC1-Y | 16.6 | 49 | 17.8 |

Data shown represent ED₅₀ values in μg/ml of the enzymes tested, as calculated by regression analysis using a polynomial regression with R² ranging from 0.93 to 0.98.

These results therefore demonstrate that the Pichia expression system of the present invention enables the large-scale production of pure CHIT36 protein, and that combined rCHIT36 and EXC1-Y chitinases possess potent and highly synergistic chitinase and antifungal activities capable of uniquely and very significantly reducing growth of the fungal phytopathogens Alternaria alternata, Botrytis cinerea and, for the first time using a preparation comprising a recombinant fungal chitinase, Fusarium oxysporum. As such this novel combination of chitinases is superior to all prior art fungal chitinase preparations comprising having anti-fungal pathogen activity, and is therefore highly suitable for use in protection of plants from chitin-containing pathogens, such as fungi.

### Example 3

### Stress-Inducible Expression of Heterologous Reporter Transgenes Under the Regulatory Control of the chit36 Endochitinase Promoter

Exposure of crops to various stress conditions, such as temperature extremes, nitrogen paucity, osmotic stress, chitin-containing pathogens, etc., are routinely involved in causing major losses in agricultural productivity. Hence, the ability to endow plants with the capacity to express genes conferring resistance to such stresses in response to exposure to such stress conditions is highly desirable.

While reducing the present invention to practice, the present inventors uncovered the capacity of the chit36 promoter to such drive such stress inducible expression of heterologous transgenes when expressed in organisms exposed to the aforementioned stresses, as follows.

Materials and Methods:

Stress-induced CHIT36 expression assays: In order to analyze the capacity of the chit36 promoter to drive stress-inducible gene expression, wild-type T. harzianum was subjected to the following treatments: growth in glucose rich medium, growth in chitin supplemented medium, 4° C. treatment, 40° C. treatment, 2% EtOH treatment, Lane 6: 1% glucosamine (a nitrogen and carbon source treatment, 1% N-acetylglucosamine (chitin degradation product/carbon and nitrogen source) treatment, and nitrogen starvation treatment. Following these treatments, chit36 transcription levels were analyzed via Northern Blot using a chit36 specific probe.

Cloning and characterization of the chit36 promoter: The genomic DNA segment comprising the 1942 bp upstream of the chit36 coding segment was isolated using the Universal GenomeWalker Kit (Clontech) according to the user manual, sequenced, and computationally analyzed for the presence of putative ORFs and gene regulatory sequences.

Reporter gene expression vector construction and transformants: For generation of a reporter gene expression vector for testing stress inducible expression of reporter genes under the regulatory control of the chit36 promoter, the 830 bp chit36 promoter was inserted upstream of gfp coding sequences in the gfp reporter gene expression vector pZEGA1 (kindly provided by Prof. C P Kubicek, Austria). T. harzianum transformants were generated with this vector via microprojectile bombardment. To determine the transgene copy number in transformants, genomic DNA thereof was digested with EcoRI, which does not cut in the gfp sequences, and analyzed via Southern Blot using a gfp specific probe (data not shown).

Reporter Gene Expression Assays:

Chitin inducible expression assays: For analysis of the chitin inducibility of chit36 promoter regulated reporter gene expression, transformants were grown in medium supplemented with 0.2% colloidal chitin and analyzed via fluorescence microscopy for GFP expression.

Pathogen confrontation assays: For analysis of the inducibility of the chit36 promoter to the presence of phytopathogens, gfp expression by T. harzianum transformants was measured via fluorescence microscopy following exposure of transformants to phytopathogens, as follows. Transformants were grown for 2 days on a cellophane or dialysis membrane disk allowing passage of cellular secretions on SM medium supplemented with 0.2% glucose, the disks were transferred to a plate where the phytopathogen R. solani was pre-grown for 1 day and either left in place or removed prior to addition of the disk, and gfp expression was visualized.

Experimental Results:

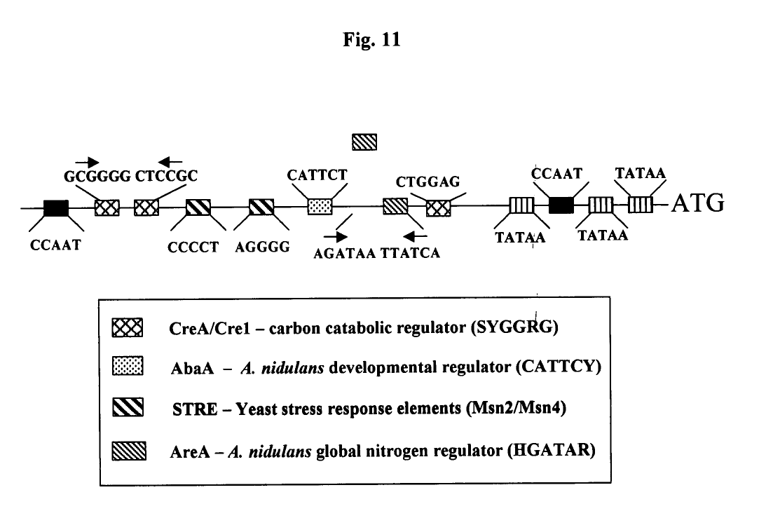

Cloning and characterization of upstream regulatory sequences of chit36: The 1939 bp segment of genomic DNA upstream of the chit36 ORF was sequenced (SEQ ID NO: 14) and unexpectedly found to encode a 144 residue polypeptide (nucleotides 678-1109 of SEQ ID NO: 14) in a different reading frame from chit36. This 144 residue protein was found to be 37% identical with a hypothetical protein from Streptomyces coecicolor (SC1B2.19). The closest homologue of CHIT36 is also a putative chitinase from Streptomyces coecicolor, as described above. Since this 144 residue protein is positioned from 1262 to 831 nucleotides upstream of chit36 coding sequences, the chit36 promoter (nucleotides 1110-1939 of SEQ ID NO: 14) was delimited to the 830 bp segment upstream of the chit36 transcription start. Analysis of this 830 bp segment (FIG. 11) revealed the presence of 3 putative TATAA (SEQ ID NO: 15) boxes, two CCAAT (SEQ ID NO: 16) boxes and consensus motifs for the CreA/Crel carbon catabolic repressor (SYGGRG (SEQ ID NO: 17)). The consensus motifs HGATAR (SEQ ID NO: 18) and CATTCY (SEQ ID NO: 19) for the binding of A. nidulans global nitrogen regulator and the AbaA developmental regulator were found at −542, −514, and at −589, respectively. As well, the yeast stress response element binding sites (Msn2/Msn4) were found to be present at −599 and −713. No sequences matching the four MYC (SEQ ID NO: 20) motifs present in the upstream regulatory sequences of both ech42 and prb1 were identified. The presence of these sequences strongly suggested that chit36 is inducible under general stress conditions. No significant homologs of the 830 bp segment comprising the chit36 upstream regulatory sequences were identified.

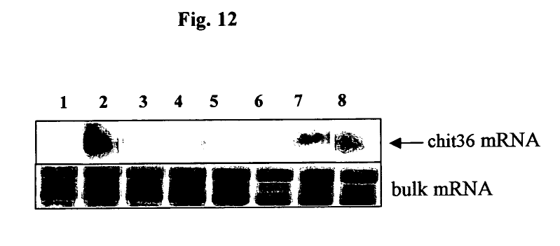

The chit36 promoter is inducible under a broad range of stress conditions: As shown in FIG. 11, high (40° C.) or low (4° C.) temperature stresses, osmotic stress (2% EtOH) or nitrogen starvation (0.1×concentration of nitrogen sources) were found to upregulate chit36 transcription. Furthermore, nitrogen starvation treatment of T. harzianum abrogated the capacity of culture in glucose rich (2%) medium to repress chit36 transcription (FIG. 12, lane 8).

Chitin induces expression of heterologous genes under the regulatory control of the chit36 promoter: Two transformants, C1 and B2, each bearing 3-4 copies of the reporter gene, were found to specifically express gfp when grown in chitin-supplemented medium but not in non-inductive (glucose-rich) medium (results not shown).

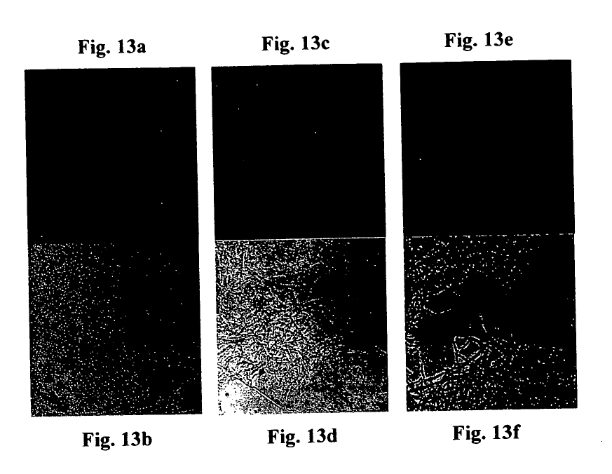

Exposure to phytopathogens induces high levels of expression of a reporter gene under the regulatory control of the chit36 promoter in transformants: As shown in FIGS. 13a-b, transformants expressing gfp under the regulatory control of the chit36 promoter expressed high levels of gfp, as determined by fluorescence microscopy, when grown on a solute-permeable disk placed on agar containing the secretions from a 2-day culture of the phytopathogen R. solani and from which the phytopathogen was removed (FIGS. 13a-b), and expressed even higher levels under similar conditions when the phytopathogen was not removed (FIGS. 13c-d). When transformants were grown over virgin agar, no gfp expression was seen (FIGS. 13e-f).

In summary, these results therefore demonstrated that the chit36 promoter can be used to drive expression of reporter genes in response to a broad range of stress conditions, including temperature extremes, nitrogen paucity, osmotic stress, and exposure to chitin or chitin-containing pathogens, such as phytopathogenic fungi. The chit36 promoter can be used, for example. to drive stress inducible expression of stress resistance genes in transgenic crop plants, thereby protecting such plants from stresses such as the aforementioned stresses.

### Example 4

### Transgenic Expression CHIT36 Chitinase in Crop Plants Confers Protection from Chitin-Containing Pathogens

Numerous chitin-containing pathogens, such as fungal, nematode and insect pathogens are routinely responsible for large-scale losses of agricultural productivity. Thus, means of protecting plants from such pathogens are highly desirable. As described above, the novel recombinant fungal endochitinase, CHIT36, of the present invention possesses potent chitinase activity and anti-fungal phytopathogen activity. Thus, in order to confer resistance to chitin-containing pathogens, to tobacco, potato and apple plants, such plants are genetically transformed to express rCHIT36 under the regulatory control of a heterologous promoter capable of driving constitutive reporter gene expression.

### Example 5

### Crop Plants Transgenic for a Stress Resistance Gene under the Regulatory Control of the chit36 Promoter Specifically Express the Stress-Resistance Gene in Response to Stress and are Protected from Stress

The capacity to endow organisms such as plants with the ability to express homologous or heterologous stress resistance genes in response to stresses is highly desirable since, for example, exposure of crop plants to various stresses, such as temperature extremes, osmotic stress, nitrogen paucity and attack or exposure to chitin-containing pathogens, are routinely responsible for causing enormous losses in agricultural productivity. Furthermore, the capacity to specifically induce expression of stress resistance genes in response under stress conditions is highly desirable since this minimizes any potential unwanted side-effects, such as growth inhibition, caused by the expression of the stress resistance gene by downregulating its transcription under non-stress conditions.

Thus, in order to endow a crop plant with protection from one of the aforementioned stress conditions, a gene conferring resistance to such a stress is expressed in the plant under the regulatory control of the chit36 promoter which, as described above, has the capacity to induce expression of a reporter gene under its regulatory control in response to all of the aforementioned stresses.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

What is claimed is:

1. An isolated polypeptide displaying an endochitinase activity and comprising an amino acid sequence being at least 84% similar to SEQ ID NO: 13 or a portion thereof, as determined using the Standard protein-protein BLAST [blastp] software of the NCBI.

2. The isolated polypeptide of claim 1, wherein said portion is amino acid residues 17 to 344 of SEQ ID NO: 13.

3. The isolated polypeptide of claim 1, wherein the isolated polypeptide is characterized by an apparent molecular weight of about 33 kDa, as determined via 12% SDS-PAGE following deglycosylation.

4. The isolated polypeptide of claim 1, wherein the isolated polypeptide is characterized by a pI selected from a range of about 4.5 to about 4.9.

5. The isolated polypeptide of claim 1, wherein said endochitinase activity is optimal at a pH of about 4.5.

6. The isolated polypeptide of claim 1, wherein said endochitinase activity is optimal at a temperature selected from a range of about 40° C. to about 53° C.

7. The isolated polypeptide of claim 1, wherein the isolated polypeptide comprises a signal peptide.

8. The isolated polypeptide of claim 7, wherein said signal peptide is for extracellular secretion of the isolated polypeptide.

9. The isolated polypeptide of claim 7, wherein said signal peptide comprises amino acid residues 1-16 of SEQ ID NO: 13.

10. The isolated polypeptide of claim 1, wherein the isolated polypeptide exhibits an activity against a chitin-containing organism.

11. The isolated polypeptide of claim 10, wherein said activity against said chitin-containing organism is selected from the group consisting of inhibition of growth of said chitin-containing organism, killing of said chitin-containing organism and inhibition of reproduction of said chitin-containing organism.

12. The isolated polypeptide of claim 10, wherein said chitin-containing organism is a fungus.

13. The isolated polypeptide of claim 12, wherein said fungus is selected from the group consisting of Botrytis cinerea, Fusarium oxysporum, Sclerotium rolfsii and Candida albicans.

14. A pharmaceutical composition comprising as an active ingredient a polypeptide comprising an amino acid sequence being at least 84% similar to SEQ ID NO: 13 or a portion thereof, as determined using the Standard protein-protein BLAST [blastp] software of the NCBI, and a pharmaceutically acceptable carrier or diluent, said polypeptide displaying an endochitinase activity.

15. The pharmaceutical composition of claim 14, wherein said portion is amino acid residues 17 to 344 of SEQ ID NO: 13.

16. The pharmaceutical composition of claim 14, further comprising as an active ingredient a polypeptide displaying an exochitinase activity.

17. The pharmaceutical composition of claim 16, wherein said polypeptide displaying said exoclitinase activity is EXC-1.

18. The pharmaceutical composition of claim 14, wherein said carrier or diluent is formulated for topical administration.

19. A composition for disinfesting chitin-containing organisms, the composition comprising as an active ingredient a polypeptide displaying an endochitinase activity, said polypeptide comprising an amino acid sequence being at least 84% similar to SEQ ID NO: 13 or a portion thereof, as determined using the Standard protein-protein BLAST [blastp] software of the NCBI.

20. The composition of claim 19, wherein said portion is amino acid residues 17 to 344 of SEQ ID NO: 13.

21. The composition of claim 19, wherein the composition further comprises a carrier or diluent.

22. The composition for disinfesting chitin-containing organisms, further comprising as an active ingredient a polypeptide displaying an exochitinase activity.

23. The composition of claim 22, wherein said polypeptide displaying said exochitinase activity is EXC-1.

24. An agronomic composition comprising as an active ingredient a polypeptide displaying an endochitinase activity, said polypeptide comprising an amino acid sequence being at least 84% similar to SEQ ID NO: 13 or a portion thereof, as determined using the Standard protein-protein BLAST [blastp] software of the NCBI.

25. The agronomic composition of claim 24, wherein the agronomic composition further comprises an agronomically acceptable carrier or diluent.

26. The agronomic composition of claim 24, wherein said portion is amino acid residues 17 to 344 of SEQ ID NO: 13.

27. The agronomic composition of claim 24, further comprising as an active ingredient a polypeptide displaying an exochitinase activity.

28. The agronomic composition of claim 27, wherein said polypeptide displaying said exochitinase activity is EXC-1.

29. An enzymatic composition comprising a polypeptide displaying an endochitinase activity, said polypeptide comprising an amino acid sequence being at least 84% similar to SEQ ID NO: 13 or a portion thereof, as determined using the Standard protein-protein BLAST [blastp] software of the NCBI.

30. The enzymatic composition of claim 29, wherein said portion is amino acid residues 17 to 344 of SEQ ID NO: 13.

31. The enzymatic composition of claim 29, further comprising a polypeptide displaying an exochitinase activity.

32. The enzymatic composition of claim 31, wherein said polypeptide displaying said exochitinase activity is EXC-1.

33. An isolated polynucleotide comprising a nucleic acid sequence being at least 84% identical to SEQ ID NO: 12 or a portion of SEQ ID NO: 12, as determined using the Standard nucleotide-nucleotide BLAST [blastn] software of the NCBI, said nucleic acid sequence encoding a polypeptide displaying an endochitinase activity.

34. The isolated polynucleotide of claim 33, wherein said portion of SEQ ID NO: 12 is selected from the group consisting of nucleotides 1-1138 of SEQ ID NO: 12, nucleotides 104-1345 of SEQ ID NO: 12, nucleotides 104-1138 of SEQ ID NO: 12, nucleotides 152-1345 of SEQ ID NO: 12, and nucleotides 152-1138 of SEQ ID NO: 12.

35. The isolated polynucleotide of claim 33, wherein said polypeptide is at least 84% similar to SEQ ID NO: 13 or a portion of SEQ ID NO: 13.

36. The isolated polynucleotide of claim 35, wherein said portion of SEQ ID NO: 13 is amino acid residues 17 to 344 of SEQ ID NO: 13.

37. The isolated polynucleotide of claim 33, wherein said isolated polynucleotide is selected from the group consisting of a genomic polynucleotide, a complementary polynucleotide and a composite polynucleotide.

38. A nucleic acid construct comprising a nucleic acid sequence being at least 84% identical to SEQ ID NO: 12, or a portion of SEQ ID NO: 12, as determined using the Standard nucleotide-nucleotide BLAST [blastn] software of the NCBI, said nucleic acid sequence encoding a polypeptide displaying an endochitinase activity.

39. The nucleic acid construct of claim 38, wherein said portion of SEQ ID NO: 12 is selected from the group consisting of nucleotides 1-1138 of SEQ ID NO: 12, nucleotides 104-1345 of SEQ ID NO: 12, nucleotides 104-1138 of SEQ ID NO: 12, nucleotides 152-1345 of SEQ ID NO: 12, and nucleotides 152-1138 of SEQ ID NO: 12.

40. The nucleic acid construct of claim 38, wherein said polypeptide is at least 84% similar to SEQ ID NO: 13, or a portion of SEQ ID NO: 13.

41. The nucleic acid construct of claim 40, wherein said portion of SEQ ID NO: 13 is amino acid residues 17 to 344 of SEQ ID NO: 13.

42. A host cell comprising a nucleic acid construct, said nucleic acid construct comprising a nucleic acid sequence being at least 84% identical to SEQ ID NO: 12, or a portion of SEQ ID NO: 12, as determined using the Standard nucleotide-nucleotide BLAST [blastn] software of the NCBI, said nucleic acid sequence encoding a polypeptide displaying an endochitinase activity.

43. The host cell of claim 42, wherein said portion of SEQ ID NO: 12 is selected from the group consisting of nucleotides 1-1138 of SEQ ID NO: 12, nucleotides 104-1345 of SEQ ID NO: 12, nucleotides 104-1138 of SEQ ID NO: 12, nucleotides 152-1345 of SEQ ID NO: 12, and nucleotides 152-1138 of SEQ ID NO: 12.

44. The host cell of claim 42, wherein said polypeptide is at least 84% similar to SEQ ID NO: 13, or a portion of SEQ ID NO: 13.

45. The host cell of claim 44, wherein said portion of SEQ ID NO: 13 is amino acid residues 17 to 344 of SEQ ID NO: 13.

46. The host cell of claim 42, wherein the host cell is a Trichoderma harzianum cell or a Pichia pastoris cell.

47. The host cell of claim 42, wherein the host cell is a plant cell.

48. A method of preventing or treating a disease or a condition associated with a chitin-containing organism in an individual, the method comprising administering to the individual a therapeutically effective amount of a pharmaceutical composition including as an active ingredient a polypeptide displaying an endochitinase activity and being at least 84% similar to SEQ ID NO: 13 or a portion thereof, as determined using the Standard protein-protein BLAST [blastp] software of the NCBI, said pharmaceutical composition further including a pharmaceutically acceptable carrier or diluent.

49. The method of claim 48, wherein said pharmaceutical composition further includes as an active ingredient a polypeptide displaying an exochitinase activity.

50. The method of claim 49, wherein said polypeptide displaying said exochitinase activity is EXC-1.

51. The method of claim 48, wherein the disease or condition is dermal or mucosal and further wherein said carrier or diluent is formulated for topical administration.

52. The method of claim 48, wherein said portion is amino acid residues 17 to 344 of SEQ ID NO: 13.

53. The method of claim 48, wherein the chitin-containing organism is a fungus.

54. The method of claim 48, wherein the chitin-containing organism is Candida albicans.

55. The method of claim 48, wherein the chitin-containing organism is associated with pathogenesis of the disease or condition.

56. The method of claim 48, wherein said polypeptide displaying said endochitinase activity is characterized by an apparent molecular weight of about 33 kDa, as determined via 12% SDS-PAGE following deglycosylation.

57. The method of claim 48, wherein said polypeptide displaying said endochitinase activity is characterized by a pI selected from a range of about 4.5 to about 4.9.

58. The method of claim 48, wherein said endochitinase activity is optimal at a pH of about 4.5.

59. The method of claim 48, wherein said endochitinase activity is optimal at a temperature selected from a range of about 40° C. to about 53° C.

60. The method of claim 48, wherein said polypeptide displaying said endochitinase activity comprises a signal peptide.

61. The method of claim 60, wherein said signal peptide is for extracellular secretion of said polypeptide displaying said endochitinase activity.

62. The method of claim 60, wherein said signal peptide comprises amino acid residues 1-16 of SEQ ID NO: 13.

63. The method of claim 48, wherein said polypeptide displaying said endochitinase activity exhibits an activity against the chitin-containing organism.

64. The method of claim 63, wherein said activity against the chitin-containing organism is selected from the group consisting of inhibition of growth of the chitin-containing organism, killing of the chitin-containing organism and inhibition of reproduction of the chitin-containing organism.

65. A method of preventing or treating a disease or condition associated with a chitin-containing organism in a plant, the method comprising expressing within the plant an exogenous polypeptide displaying an endochitinase activity and being at least 84% similar to SEQ ID NO: 13 or a portion thereof, as determined using the Standard protein-protein BLAST [blastp] software of the NCBI.

66. The method of claim 65, wherein the chitin-containing organism is associated with pathogenesis of the disease or condition.

67. The method of claim 65, wherein said portion is amino acid residues 17 to 344 of SEQ ID NO: 13.

68. The method of claim 65, wherein the chitin-containing organism is a fungus.

69. The method of claim 68, wherein said fungus is selected from the group consisting of Botrytis cinerea, Fusarium oxysporum and Sclerotium rolfsii.

70. The method of claim 68, wherein said exogenous polypeptide displaying said endochitinase activity is characterized by an apparent molecular weight of about 33 kDa, as determined via 12% SDS-PAGE following deglycosylation.

71. The method of claim 65, wherein said exogenous polypeptide is characterized by a pI selected from a range of about 4.5 to about 4.9.

72. The method of claim 65, wherein said endochitinase activity is optimal at a pH of about 4.5.

73. The method of claim 65, wherein said endochitinase activity is optimal at a temperature selected from a range of about 40° C. to about 53° C.

74. The method of claim 65, wherein said exogenous polypeptide comprises a signal peptide.

75. The method of claim 74, wherein said signal peptide is for extracellular secretion of said exogenous polypeptide.

76. The method of claim 74, wherein said signal peptide comprises amino acid residues 1-16 of SEQ ID NO: 13.

77. The method of claim 65, wherein said exogenous polypeptide exhibits an activity against the chitin-containing organism.

78. The method of claim 77, wherein said activity against the chitin-containing organism is selected from the group consisting of inhibition of growth of the chitin-containing organism, killing of the chitin-containing organism and inhibition of reproduction of the chitin-containing organism.

79. A method of preventing or treating a disease or condition associated with a chitin-containing organism in a plant, the method comprising contacting the plant with a composition including as an active ingredient a polypeptide displaying an endochitinase activity and being at least 84% similar to SEQ ID NO: 13 or a portion thereof, as determined using the Standard protein-protein BLAST [blastp] software of the NCBI.

80. The method of claim 79, wherein the chitin-containing organism is associated with pathogenesis of the disease or condition.

81. The method of claim 79, wherein said composition further includes a diluent.

82. The method of claim 79, wherein said portion is amino acid residues 17 to 344 of SEQ ID NO: 13.

83. The method of claim 79, wherein said composition further includes as an active ingredient a polypeptide displaying an exochitinase activity.

84. The method of claim 83, wherein said said polypeptide displaying said exochitinase activity is EXC-1.

85. The method of claim 79, wherein the chitin-containing organism is a fungus.

86. The method of claim 85, wherein said fungus is selected from the group consisting of Botrytis cinerea, Fusarium oxysporum and Sclerotium rolfsii.

87. The method of claim 79, wherein said polypeptide displaying said endochitinase activity is characterized by an apparent molecular weight of about 33 kDa, as determined via 12% SDS-PAGE following deglycosylation.

88. The method of claim 79, wherein said polypeptide displaying said endochitinase activity is characterized by a pI selected from a range of about 4.5 to about 4.9.

89. The method of claim 79, wherein said endochitinase activity is optimal at a pH of about 4.5.

90. The method of claim 79, wherein said endochitinase activity is optimal at a temperature selected from a range of about 40° C. to about 53° C.

91. The method of claim 79, wherein said polypeptide displaying said endochitinase activity comprises a signal peptide.

92. The method of claim 91, wherein said signal peptide is for extracellular secretion of said polypeptide displaying said endochitinase activity.

93. The method of claim 91, wherein said signal peptide comprises amino acid residues 1-16 of SEQ ID NO: 13.

94. The method of claim 79, wherein said polypeptide displaying said endochitinase activity exhibits an activity against the chitin-containing organism.

95. The method of claim 94, wherein said activity against the chitin-containing organism is selected from the group consisting of inhibition of growth of the chitin-containing organism, killing of the chitin-containing organism and inhibition of reproduction of the chitin-containing organism.

96. A method of preventing or reducing susceptibility of a plant to cold damage, the method comprising expressing within the plant an exogenous polypeptide displaying an endochitinase activity and being at least 84% similar to SEQ ID NO: 13 or a portion thereof, as determined using the Standard protein-protein BLAST [blastp] software of the NCBI.

97. The method of claim 96, wherein said portion is amino acid residues 17 to 344 of SEQ ID NO: 13.

98. A plant, a plant tissue or a plant seed comprising an exogenous polynucleotide, said exogenous polynucleotide comprising a nucleic acid sequence being at least 84% identical to SEQ ID NO: 12 or a portion of SEQ ID NO: 12, as determined using the Standard nucleotide-nucleotide BLAST [blastn] software of the NCBI, said nucleic acid sequence encoding a polypeptide displaying an endochitinase activity.

99. The plant, plant tissue or plant seed of claim 98, wherein said portion of SEQ ID NO: 12 is selected from the group consisting of nucleotides 1-1138 of SEQ ID NO: 12, nucleotides 104-1345 of SEQ ID NO: 12, nucleotides 104-1138 of SEQ ID NO: 12, nucleotides 152-1345 of SEQ ID NO: 12, and nucleotides 152-1138 of SEQ ID NO: 12.

100. The plant, plant tissue or plant seed of claim 98, wherein said polypeptide displaying said endochitinase activity is at least 84% similar to SEQ ID NO: 13, or a portion of SEQ ID NO: 13.

101. The plant, plant tissue or plant seed of claim 100, wherein said portion of SEQ ID NO: 13 is amino acid residues 17 to 344 of SEQ ID NO: 13.

102. The plant, plant tissue or plant seed of claim 98, wherein said exogenous polynucleotide is selected from the group consisting of a genomic polynucleotide, a complementary polynucleotide and a composite polynucleotide.

103. An isolated polynucleotide comprising a nucleic acid sequence being at least 30% identical to SEQ ID NO: 14 or a portion thereof, as determined using the Standard nucleotide-nucleotide BLAST [blastn] software of the NCBI, said nucleic acid sequence being capable of inducing a specific change in a level of expression of a reporter gene under the regulatory control of said nucleic acid sequence in a cell in response to exposure of said cell to a specific environmental condition.

104. The isolated polynucleotide of claim 103, wherein said portion is nucleotides 1110-1139 of SEQ ID NO: 14.

105. The isolated polynucleotide of claim 103, wherein said specific environmental condition is a stress condition and whereas said specific change is an increase in said level of expression of said reporter gene.

106. The isolated polynucleotide of claim 105, wherein said stress condition is selected from the group consisting of a temperature extreme, an elevated chitin concentration, a chitin-containing organism, osmotic stress and nitrogen starvation.

107. The isolated polynucleotide of claim 106, wherein said temperature extreme is a temperature no greater than about 4° C. or a temperature no lower than about 40° C.

108. The isolated polynucleotide of claim 106, wherein said elevated chitin concentration is no lower than about 2 g/L.

109. The isolated polynucleotide of claim 103, wherein said specific environmental condition is an elevated glucose concentration and whereas said specific change is a decrease in said level of expression of said reporter gene.

110. The isolated polynucleotide of claim 109, wherein said elevated glucose concentration is no lower than about 50 g/L.

111. The isolated polynucleotide of claim 103, wherein said nucleic acid sequence is a promoter, an enhancer or a suppressor.

112. The isolated polynucleotide of claim 103, wherein said reporter gene encodes a structural sequence of chit36 or gfp.

113. The isolated polynucleotide of claim 103, wherein said cell is a T. harzianum cell or a P. pastoris cell.

114. The isolated polynucleotide of claim 103, wherein said cell is a plant cell.

115. A nucleic acid construct comprising a nucleic acid sequence being at least 30% identical to SEQ ID NO: 14, or a portion thereof, as determined using the Standard nucleotide-nucleotide BLAST [blastn] software of the NCBI, said nucleic acid sequence being capable of inducing a specific change in a level of expression of a reporter gene under the regulatory control of said nucleic acid sequence in a cell in response to exposure of said cell to a specific environmental condition.

116. The nucleic acid construct of claim 115, wherein said portion is nucleotides 1110-1139 of SEQ ID NO: 14.

117. The nucleic acid, construct of claim 115, wherein said nucleic acid sequence is a promoter, an enhancer or a suppressor.

118. A host cell comprising a nucleic acid construct, said nucleic acid construct comprising a nucleic acid sequence being at least 30% identical to SEQ ID NO: 14, or a portion thereof, as determined using the Standard nucleotide-nucleotide BLAST [blastn] software of the NCBI, said nucleic acid sequence being capable of inducing a specific change in a level of expression of a reporter gene under the regulatory control of said nucleic acid sequence in a cell in response to exposure of said cell to a specific environmental condition.

119. The host cell of claim 118, wherein said portion is nucleotides 1110-1139 of SEQ ID NO: 14.

120. The host cell of claim 118, wherein the host cell is a Trichoderma harzianum cell or a Pichia pastoris cell.

121. The host cell of claim 118, wherein the host cell is a plant cell.

122. A plant, a plant tissue or a plant seed comprising an exogenous polynucleotide, said exogenous polynucleotide comprising a nucleic acid sequence being at least 30% identical to SEQ ID NO: 14 or a portion thereof, as determined using the Standard nucleotide-nucleotide BLAST [blastn] software of the NCBI, said nucleic acid sequence being capable of inducing a specific change in a level of expression of a reporter gene under the regulatory control of said nucleic acid sequence in a cell in response to exposure of said cell to a specific environmental condition.

123. The plant, plant tissue or plant seed of claim 122, wherein said portion is nucleotides 1110-1139 of SEQ ID NO: 14.

124. The plant, plant tissue or plant seed of claim 122, wherein said nucleic acid sequence is a promoter, an enhancer or a suppressor.

125. A method of inducing a specific change in a level of expression of a gene product in a cell in response to an exposure of the cell to a specific environmental condition, the method comprising expressing the gene product in the cell under the regulatory control of an exogenous polynucleotide comprising a nucleic acid sequence being at least 30% identical to SEQ ID NO: 14 or a portion thereof, as determined using the Standard nucleotide-nucleotide BLAST [blastn] software of the NCBI.

126. The method of claim 125, wherein said portion is nucleotides 1110-1139 of SEQ ID NO: 14.

127. The method of claim 125, wherein the gene product is endogenous or exogenous to the cell.

128. The method of claim 125, wherein the specific environmental condition is a stress condition and whereas the specific change is an increase in the level of expression of the gene product.

129. The method of claim 128, wherein said stress condition is selected from the group consisting of a temperature extreme, an elevated chitin concentration, a chitin-containing organism, osmotic stress and nitrogen starvation.

130. The method of claim 129, wherein said temperature extreme is a temperature no greater than about 4° C. or a temperature no lower than about 40° C.

131. The method of claim 129, wherein said elevated chitin concentration is no lower than about 2 g/L.

132. The method of claim 125, wherein the specific environmental condition is an elevated glucose concentration and whereas the specific change is a decrease in the level of expression of the gene product.

133. The method of claim 132, wherein said elevated glucose concentration is no lower than about 50 g/L.

134. The method of claim 125, wherein said nucleic acid sequence is a promoter, an enhancer or a suppressor.

135. The method of claim 125, wherein the gene product is a messenger RNA or a polypeptide.

136. The method of claim 125, wherein the gene product is a chit36 gene product or a gfp gene product.

137. The method of claim 125, wherein the cell is a T. harzianum cell or a P. pastoris cell.

138. The method of claim 125, wherein the cell is a plant cell.

139. A method of reducing the susceptibility of a plant to a damage resulting from an exposure to a specific environmental condition, the method comprising expressing a gene product in the plant-under the regulatory control of an exogenous polynucleotide comprising a nucleic acid sequence being at least 30% identical to SEQ ID NO: 14 or a portion thereof, as determined using the Standard nucleotide-nucleotide BLAST [blastn] software of the NCBI, said gene product being capable of reducing the susceptibility of the plant to the damage resulting from the exposure to the specific environmental condition.

140. The method of claim 139, wherein said portion is nucleotides 1110-1139 of SEQ ID NO: 14.

141. The method of claim 139, wherein said gene product is endogenous or exogenous to the plant.

142. The method of claim 139, wherein the specific environmental condition is a stress condition or an elevated glucose concentration.

143. The method of claim 142, wherein said stress condition is selected from the group consisting of a temperature extreme, an elevated chitin concentration, a chitin-containing organism, osmotic stress and nitrogen starvation.

144. The method of claim 143, wherein said temperature extreme is a temperature no greater than about 4° C. or a temperature no lower than about 40° C.

145. The method of claim 143, wherein said elevated chitin concentration is no lower than about 2 g/L.

146. The method of claim 142, wherein said elevated glucose concentration is no lower than about 50 g/L.

147. The method of claim 139, wherein said nucleic acid sequence is a promoter, an enhancer or a suppressor.

148. The method of claim 139, wherein said gene product is a messenger RNA or a polypeptide.

149. The method of claim 148, wherein said polypeptide displays an endochitinase activity and comprises an amino acid sequence being at least 84% similar to SEQ ID NO: 13 or a portion thereof, as determined using the Standard protein-protein BLAST [blastp] software of the NCBI.

150. The method of claim 149, wherein said portion is amino acid residues 17 to 344 of SEQ ID NO: 13.
